SEQUENCE LISTING (27 sequences)

SEQ ID NO: 1 - protein, 8 aa, Artificial Sequence
  peptide
  MISC_FEATURE (1)..(1): X is a hydrophobic amino acid
  misc_feature (2)..(2): Xaa can be any naturally occurring amino acid
  MISC_FEATURE (3)..(3): X is a non-natural analogue of phosphotyrosine, tyrosine or phosphotyrosine
  MISC_FEATURE (4)..(4): X is a hydrophobic amino acid
  MISC_FEATURE (5)..(5): X is an anionic amino acid
  MISC_FEATURE (6)..(6): X is an hydrophobic amino acid
  MISC_FEATURE (7)..(7): X is an anionic amino acid
  MISC_FEATURE (8)..(8): X is an hydrophobic amino acid
XXXXXXXX

SEQ ID NO: 2 - protein, 9 aa, Artificial Sequence
  peptide
  MOD_RES (4)..(4): PHOSPHORYLATION
GLNYIDLDL

SEQ ID NO: 3 - protein, 13 aa, Artificial Sequence
  peptide
  MOD_RES (7)..(7): PHOSPHORYLATION
GDKQVEYLDLDLD

SEQ ID NO: 4 - protein, 8 aa, Artificial Sequence
  peptide
  MISC_FEATURE (3)..(3): X is phosphonodifluoromethyl phenylalanine
LNXIDLDW

SEQ ID NO: 5 - protein, 9 aa, Artificial Sequence
  peptide
  MISC_FEATURE (4)..(4): X is phosphonodifluoromethyl phenylalanine
GLNXIDLDW

SEQ ID NO: 6 - protein, 11 aa, Artificial Sequence
  peptide
  MOD_RES (6)..(6): PHOSPHORYLATION
DKQVEYLDLDL

SEQ ID NO: 7 - protein, 11 aa, Artificial Sequence
  peptide
  MOD_RES (6)..(6): PHOSPHORYLATION
EEENIYSVPHD

SEQ ID NO: 8 - protein, 11 aa, Artificial Sequence
  peptide
  MOD_RES (6)..(6): PHOSPHORYLATION
VDADEYLIPQQ

SEQ ID NO: 9 - protein, 38 aa, Artificial Sequence
  peptide
  MOD_RES (8)..(8): PHOSPHORYLATION
  MOD_RES (32)..(32): PHOSPHORYLATION
GGGGDITYADLNLPKGKKPAPQAAEPNNHTEYASIQTS

SEQ ID NO: 10 - protein, 20 aa, Artificial Sequence
  peptide
  MISC_FEATURE (1)..(1): X is 5,6 carboxyfluorescein
  MOD_RES (5)..(5): PHOSPHORYLATION
XGLNYIDLDWGRKKRRQRRR

SEQ ID NO: 11 - protein, 21 aa, Artificial Sequence
  transportan 10
AGYLLGKINLKALAALAKKIL

SEQ ID NO: 12 - protein, 9 aa, Artificial Sequence
  peptide
GLNYIDLDL

SEQ ID NO: 13 - protein, 8 aa, Artificial Sequence
  peptide
  MOD_RES (3)..(3): PHOSPHORYLATION
LNYIDLDL

SEQ ID NO: 14 - protein, 7 aa, Artificial Sequence
  peptide
  MOD_RES (2)..(2): PHOSPHORYLATION
NYIDLDL

SEQ ID NO: 15 - protein, 8 aa, Artificial Sequence
  peptide
  MOD_RES (3)..(3): PHOSPHORYLATION
LNYIDLDW

SEQ ID NO: 16 - protein, 8 aa, Artificial Sequence
  peptide
  MOD_RES (3)..(3): PHOSPHORYLATION
LNYIDLDF

SEQ ID NO: 17 - protein, 8 aa, Artificial Sequence
  peptide
  MOD_RES (3)..(3): PHOSPHORYLATION
LNYIDLEW

SEQ ID NO: 18 - protein, 10 aa, Artificial Sequence
  peptide
  MOD_RES (1)..(1): X is 5,6 carboxyfluorescein
  MOD_RES (5)..(5): PHOSPHORYLATION
XGLNYIDLDL

SEQ ID NO: 19 - protein, 10 aa, Artificial Sequence
  peptide
  MISC_FEATURE (1)..(1): X is 5,6 carboxyfluorescein
XGLNYIDLDL

SEQ ID NO: 20 - protein, 10 aa, Artificial Sequence
  peptide
  misc_feature (1)..(1): X is 5,6 carboxyfluorescein
  MOD_RES (5)..(5): PHOSPHORYLATION
XGLNYIDLDW

SEQ ID NO: 21 - protein, 10 aa, Artificial Sequence
  peptide
  MISC_FEATURE (1)..(1): X is cyanine 3
  MOD_RES (5)..(5): PHOSPHORYLATION
XGLNYIDLDW

SEQ ID NO: 22 - protein, 10 aa, Artificial Sequence
  peptide
  MISC_FEATURE (1)..(1): X is 5,6 carboxyfluorescein
  MOD_RES (5)..(5): PHOSPHORYLATION
XGLNYIDLEW

SEQ ID NO: 23 - protein, 10 aa, Artificial Sequence
  peptide
  MISC_FEATURE (1)..(1): X is 5,6 carboxyfluorescein
  MISC_FEATURE (5)..(5): X is (CF2PO3)Phe
XGLNXIDLDW

SEQ ID NO: 24 - protein, 18 aa, Artificial Sequence
  peptide
  MOD_RES (3)..(3): PHOSPHORYLATION
LNYIDLDWGRKKRRQRRR

SEQ ID NO: 25 - protein, 10 aa, Artificial Sequence
  TAT
GRKKRRQRRR

SEQ ID NO: 26 - protein, 18 aa, Artificial Sequence
  penetratin
RQIKIWFQNRRMKWKKGG

SEQ ID NO: 27 - protein, 21 aa, Artificial Sequence
  Pep1
KETWWETWWTEWSQPKKKRKV

(19) United States
(12) Patent Application Publication
(10) Pub. No.: US 2023/0192765 A1
(43) Pub. Date: Jun. 22, 2023

(54) PEPTIDES TARGETING SHP2 AND USES THEREOF

(71) Applicants: UNIVERSITÀ DEGLI STUDI DI ROMA "TOR VERGATA", Rome (RM) (IT); OSPEDALE PEDIATRICO BAMBINO GESÙ, Rome (RM) (IT)

(72) Inventors: Lorenzo STELLA, Rome (RM) (IT); Barbara BIONDI, Rome (RM) (IT); Gianfranco BOCCHINFUSO, Rome (RM) (IT); Simone MARTINELLI, Rome (RM) (IT); Marco TARTAGLIA, Rome (RM) (IT)

(73) Assignees: UNIVERSITÀ DEGLI STUDI DI ROMA "TOR VERGATA", Rome (RM) (IT); OSPEDALE PEDIATRICO BAMBINO GESÙ, Rome (RM) (IT)

(21) Appl. No.: 17/801,044

(22) PCT Filed: Mar. 5, 2021
(86) PCT No.: PCT/EP2021/055624

§ 371 Date: Aug. 19, 2022

(30) Foreign Application Priority Data
Mar. 6, 2020 (IT) 102020000004849

(51) Int. Cl. C07K 7/06; A61K 47/64

(52) U.S. Cl. CPC C07K 7/06; A61K 47/6455

(57) ABSTRACT

The present invention relates to a peptide having the sequence from N-terminus to C-terminus X₋₂X₋₁ZX₁X₂X₃X₄X₅ wherein
Z is tyrosine, phosphotyrosine or a non-natural analogue of phosphotyrosine, such as phosphonodifluoromethyl phenylalanine (F₂Pmp)
X₋₂ is a hydrophobic amino acid, such as Leu, Ile, Val, Phe, Tyr, Trp and Met
X₋₁ is any amino acid
X₁ is a hydrophobic amino acid, such as Ile, Leu, Val, Phe, Tyr, Trp and Met
X₃ is a hydrophobic amino acid, such as Leu, Ile, Val, Phe, Tyr, Trp and Met
X₅ is a hydrophobic amino acid, such as Trp, Ile, Val, Phe, Tyr, and Met
X₂ and X₄ are anionic amino acids, preferably each independently is Asp or Glu.
The peptide inhibits protein-protein interactions of the Src homology 2 domain-containing phosphatase 2 (SHP2), for the treatment of cancer and RASopathies and as a biomedical research tool.

### TECHNICAL FIELD

The present invention refers to peptides, and derivatives thereof, and peptidomimetic inhibitors of protein-protein interactions of the SHP2 phosphatase for the treatment of cancer and RASopathies and as a biomedical research tools.

### BACKGROUND ART

### SHP2 in Physiology and Pathology

Tyrosine phosphorylation, regulated by protein-tyrosine kinases (PTKs) and protein-tyrosine phosphatases (PTPs), is a fundamental mechanism of cell signaling. Aberrant tyrosine phosphorylation, caused by hyperactive PTKs, occurs in many malignancies and most current targeted anticancer drugs are PTK inhibitors [Wu 2015]. PTPs counteract the effects of kinases, and therefore they are generally considered as negative regulators of cell signaling and as tumor suppressors [Elson 2018]. The Src homology 2 (SH2) domain-containing phosphatase 2 (SHP2), encoded by the PTPN11 gene, is a non-receptor PTP that does not conform to this simplistic picture [Tajan 2015].

SHP2 is ubiquitously expressed and mediates signal transduction downstream of various receptor tyrosine kinases (RTKs): it is required for full and sustained activation of the RAS-MAP kinase pathway [Saxton 1997], and modulates signaling also through the PI3K-AKT and JAK-STAT pathways, among others. Therefore, it is involved in the regulation of multiple cell processes, including proliferation, survival, differentiation and migration [Tajan 2015]. Considering these functions, it is not surprising that SHP2 plays a pivotal role in cancer and in developmental disorders [Grossmann 2010].

PTPN11 was the first proto-oncogene encoding a tyrosine phosphatase to be identified [Tartaglia 2003]. Somatically acquired, gain of function mutations in PTPN11 are the major cause of sporadic juvenile myelomonocytic leukemia (JMML), accounting for approximately 35% of cases [Tartaglia 2003]. JMML is a rare and aggressive myelodysplastic/myeloproliferative disorder of early childhood with a very poor prognosis, for which no drugs are presently available [Niemeyer 2019]. PTPN11 mutations also occur in childhood myelodysplastic syndromes, acute monocytic leukemia (AMoL, FAB M5) and acute lymphoblastic leukemia (ALL, “common” subgroup) [Tartaglia 2004; Grossmann 2010]. More rarely, activating mutations in this gene are found in adult myelodysplastic syndromes, chronic myelomonocytic leukemia, as well as solid tumors, including neuroblastoma, glioma, embryonal rhabdomyosarcoma, lung cancer, colon cancer and melanoma [Bentires-Alj 2004, Loh 2005, Martinelli 2006 and 2009].

In addition to malignancies driven by PTPN11 mutations, several forms of cancer are linked to the activity of wild type (WT) SHP2, too. By screening hundreds of cancer cell lines with a shRNA library, a recent groundbreaking study showed that SHP2 is required for survival of receptor tyrosine kinases (RTK)-driven cancer cells [Chen 2016]. SHP2 is also a central node in intrinsic and acquired resistance to targeted cancer drugs [Prahallad 2015; Dardarei 2018; Fedele 2018; Ruess 2018; Wong 2018; Ahmed 2019; Hill 2019; Lu 2019], which is often caused by RTK activation through feedback loops.

SHP2 is also a mediator of immune checkpoint pathways, such as PD-1 and SIRPα/CD47 [Okazaki 2013, Veillette 2018]. These signaling cascades inhibit the activation of immune cells, thus allowing self-tolerance and modulation of the duration and amplitude of physiological immune responses. SHP2 binds to the activated receptors, and is responsible for starting the signaling cascade that prevents immune cell activation [Okazaki 2013, Veillette 2018]. Some cancer cells are able to hijack these signaling pathways, thus evading antitumor immune defenses [Pardoll 2012]; therefore, SHP2 is currently being considered as a possible target for cancer immunotherapy [Ran 2016, Quintana 2019, Zhao 2019].

Finally, it is worth mentioning that induction of gastric carcinoma by H.pylori is mediated by the interaction of its virulence factor CagA with SHP2, causing aberrant activation of the phosphatase [Higashi 2002, Hayashi 2017].

In addition to its role in cancer, SHP2 is involved in a family of rare diseases collectively known as RASopathies: germline missense mutations in PTPN11 occur in ∼50% of individuals affected by Noonan syndrome (NS) [Tartaglia 2001], a JMML-prone developmental disorder [Tartaglia 2010; Roberts 2013], and in ~90% of patients affected by the related Noonan syndrome with multiple lentigines (NSML, formerly known as LEOPARD syndrome) [Digilio 2002, Legius 2002, Tajan 2018]. RASopathies are characterized by congenital cardiac anomalies, hypertrophic cardiomyopathy, short stature, musculoskeletal anomalies, dysmorphisms, variable intellectual disability and susceptibility to certain malignancies [Tartaglia 2010]. To date, the only treatment for NS and related disorders that has been explored is growth hormone therapy to improve linear growth [Roberts 2013, Gelb 2015].

### Structure and Allosteric Regulation of SHP2

The structure of SHP2 includes two Src homology 2 (SH2) domains, called N-SH2 and C-SH2, followed by the catalytic PTP domain, and an unstructured C-terminal tail with a still uncharacterized function (FIG. 1) [TaJanuary 2015]. SH2 domains are recognition elements that bind protein sequences containing a phosphorylated tyrosine (pY); in SHP2, they mediate association to RTKs, cytokine receptors, cell adhesion molecules and scaffolding adaptors [TaJanuary 2015]. Therefore, SHP2 (together with the closely related SHP1) is recruited (through its SH2 domains) by motifs containing two pYs, and dephosphorylates other (or even the same) pYs through its PTP domain.

The crystallographic structures of SHP2 [Hof 1998, LaRochelle 2018], complemented by biochemical analyses [Bocchinfuso 2007], have elucidated the main features of the allosteric regulation of SHP2 activity. Under basal conditions, the N-SH2 domain blocks the active site of the PTP domain, inserting a loop (DE or “blocking” loop) in the catalytic pocket. Consistently, the basal activity of SHP2 is very low. Association of SHP2 to its binding partners through the SH2 domains favors the release of this autoinhibitory interaction, making the catalytic site available to substrates, and causing activation (FIG. 1). Specifically, structures of the N-SH2 domain associated to phosphopeptide sequences show that association to binding partners induces a conformational change in the blocking loop, which loses complementarity to the active site [Lee 1994]. At the same time, the N-SH2/PTP interaction allosterically controls the conformation of the N-SH2 domain binding site. Structures of the autoinhibited protein show that the binding site of the N-SH2 domain is closed by two loops (EF and BG). By contrast, in structures of the isolated N-SH2 domain [Lee 1994], or the recently reported structure of the active state of SHP2 [LaRochelle 2018], the binding site is open (FIG. 1). As a consequence, it has been hypothesized that the transition between the closed, autoinhibited state and the open, active conformation is coupled to an increased affinity for binding partners [Keilhack 2005; Bocchinfuso 2007, Martinelli 2008, LaRochelle 2018].

The spectrum of pathogenic PTPN11 mutations is generally consistent with this picture of SHP2 regulation. Most mutations cluster at the N-SH2/PTP interface, destabilizing the interaction between these two domains and causing constitutive activation of the phosphatase [Tartaglia 2006; Bocchinfuso 2007]. These mutations concomitantly induce an increased responsiveness to activation by association of biphosphorylated sequences to the SH2 domains [Keilhack 2005; Bocchinfuso 2007, Martinelli 2008, LaRochelle 2018]. Other mutations localize in the binding site of the SH2 domains, and simply cause an increased affinity for phosphorylated binding partners [Tartaglia 2006]. The final effect is an upregulation of the RAS/MAPK signal transduction pathway.

### SHP2 as a Pharmacological Target

All the findings reported above clearly indicate SHP2 as an important molecular target for cancer and RASopathies [Butterworth 2014; Ran 2016; Frankson 2017].

Research efforts in SHP2-targeted drug discovery have been focused mainly on active-site inhibitors. Several molecules inhibiting the catalytic activity of SHP2 have been reported [Butterworth 2014], but many of them are affected by the same limitations that led PTPs in general to be considered “undruggable” [Blaskovich 2009; Ran 2016], i.e. lack of target specificity and poor bioavailability. Some compounds with good affinity and apparent selectivity have been described, and data supporting the potential anticancer efficacy of some SHP2 PTP inhibitors have been reported [Bunda 2015; Grosskopf 2015]. However, more recent studies demonstrated that these molecules have several off target effects [Tsutsumi 2018].

An alternative pharmacological strategy has been pursued by researchers at Novartis [Chen 2016], followed by others [Xie 2017; Nichols 2018; Wu 2019], who reported allosteric inhibitors stabilizing the autoinhibited structure of SHP2 by binding to a pocket located at the interdomain interface in the closed conformation of the phosphatase.

SHP099, the inhibitor developed by Novartis, is finding promising applications in the treatment of RTK-driven cancers [Chen 2016], KRAS and BCR-ABL1 dependent tumors [Gu 2018; Mainardi 2018; Ruess 2018; Wong 2018] and in combined therapy against drug resistant cells [Dardarei 2018; Fedele 2018; Ahmed 2019; Lu 2019]. However, this compound is generally ineffective in the case of activating PTPN11 mutants, since the allosteric binding site is lost in the open conformation of the enzyme [Sun 2018; LaRochelle 2018].

### Inhibition of Protein-Protein Interactions as an Alternative Pharmacological Strategy

Due to the allosteric mechanism described above, SHP2 activation and its association to binding partners are coupled events. Therefore, the effect of NS- and leukemia-causing mutations destabilizing the autoinhibited conformation is twofold: they cause an increase in the phosphatase activity of the protein, but at the same time favor the N-SH2 conformation suitable for binding phosphorylated proteins, thus increasing the overall responsiveness of SHP2 to its interaction partners. Several lines of evidence indicate that the second event, rather than the enhanced basal activity, is essential for the abnormal activation of the RAS/MAPK pathway.

Some pathogenic mutations, such as the NS-associated p.T42A, simply increase the binding affinity of the N-SH2 domain, without causing basal activation [Martinelli 2008, Keilhack 2005]; on the other hand, the ability of SHP2 to associate to binding partners is preserved in all the disease-associated PTPN11 mutations [Tartaglia 2006, Martinelli 2012, 2020].

Truncated constructs with deletion or partial deletion of the N-SH2 domain cause a dramatic increase in the enzymatic activity of SHP2 and, at the same time, a complete loss of its ability to bind signaling partners. These constructs were not pathogenic in heterozygous mice [Saxton 1997], and did not cause any aberrant phenotype in cells [Saxton 1997, Higashi 2002]. Indeed, RAS/MAPK signaling in homozygous cells with the mutated construct was reduced with respect to the WT cells [Shi 1998]. However, cellular morphological changes (hummingbird phenotype) were observed when the truncated construct was targeted to cellular membranes by adding a membrane-localization signal [Higashi 2002], demonstrating the importance of proper cellular localization, normally mediated by SH2 domain binding. The relevance of SHP2 association to its binding partners for its role in aberrant signaling has been demonstrated also by a study on monobodies targeting the N-SH2 domain and disrupting its association with adaptor proteins. Expression of these monobodies in cancer cells carrying the activating p.V45L mutation almost completely abolished ERK½ phosphorylation [Sha 2013].

An example of the opposite situation, where binding is preserved and the catalytic activity is impaired, is provided by PTPN11 mutations causing NSML, such as p.T468M. These mutations are located in proximity of the PTP active site, at the PTP/N-SH2 interface, and have a twofold effect: they destabilize the closed state of the protein, and as consequently promote SHP2 association to signaling partners; at the same time, they perturb the active site and therefore strongly impair the catalytic activity of the phosphatase. Interestingly, the phenotype of NSML is very similar to that of NS, and these mutations still allow the activation of multiple effector pathways, including the RAS/MAPK cascade [Martinelli 2008].

Overall, these findings strongly suggest that a mere enhancement in SHP2 catalytic activity is not sufficient to cause disease and indicate that increased association to binding partners plays a major role in the mechanism of pathogenicity of SHP2 lesions. Therefore, inhibition of SHP2 binding to other proteins through its SH2 domains represents a promising alternative pharmaceutical strategy. Although SH2 domains in general have received much attention as potential targets of pharmaceuticals [Machida 2005], no molecules targeting the SH2 domains of SHP2 for therapeutic purposes have been developed.

Kertesz and coworkers [Kertesz 2006a and b] reported that the natural SHP2-binding motif of Gab1 [GDKQVEpYLDLDLD (SEQ ID NO:3)], when delivered into immune cells, modulated phosphorylation patterns. However, this peptide contains a normal pY and standard amino acids and the authors demonstrated that it is rapidly degraded in cells. Therefore it cannot be used for therapeutic purposes as such.

There is therefore a need for novel molecules inhibiting SHP2 protein-protein interactions for new targeted therapies.

### SUMMARY OF THE INVENTION

The authors developed peptide-based molecules with low nM affinity to the N-SH2 domain of SHP2, good cell uptake and resistance to degradation, to inhibit SHP2 protein-protein interactions. The present invention provides a novel route for SHP2-targeted therapies and offers a new tool to investigate the role of protein-protein interactions in the function of SHP2.

### DESCRIPTION OF THE INVENTION

It is therefore an object of the invention a peptide comprising or consisting of the sequence from N-terminus to C-terminus:
X₋₂X₋₁ZX₁X₂X₃X₄X₅
wherein
Z is tyrosine, phosphotyrosine (pY) or a non-natural analogue of phosphotyrosine, such as phosphonodifluoromethyl phenylalanine (F₂Pmp)
X₋₂ is a hydrophobic amino acid, preferably selected from the group consisting of: Leu, Ile, Val, Phe, Tyr, Trp and Met
X₋₁ is any amino acid
X₁ is a hydrophobic amino acid, preferably selected from the group consisting of: Ile, Leu, Val, Phe, Tyr, Trp and Met
X₃ is a hydrophobic amino acid, preferably selected from the group consisting of: Leu, Ile, Val, Phe, Tyr, Trp and Met
X₅ is a hydrophobic amino acid, preferably selected from the group consisting of: Trp, Ile, Val, Phe, Tyr, and Met
X₂ and X₄ are anionic amino acids, preferably each independently is Asp or Glu wherein the amino acids may be each independently natural or non-natural amino acids, such as Cα or N methylated, peptoids, beta amino acids or D amino acids,
with the proviso that the hydrophobic amino acid in X₅ is not Leu.

Preferably, the peptide of the invention is not GLNpYIDLDL (SEQ ID NO:2) and GDKQVEpYLDLDLD (SEQ ID NO:3).

It is a further object of the invention a peptide comprising or consisting of the sequence from N-terminus to C-terminus:

X₋₂X₋₁ZX₁X₂X₃X₄X₅ (SEQ ID NO:1)

wherein
   Z is a non-natural analogue of phosphotyrosine, such as phosphonodifluoromethyl phenylalanine (F₂Pmp), tyrosine or phosphotyrosine (pY)
   X₋₂ is a hydrophobic amino acid, preferably selected from the group consisting of: Leu, Ile, Val, Phe, Tyr, Trp and Met
   X₋₁ is any amino acid
   X₁ is a hydrophobic amino acid, preferably selected from the group consisting of: Ile, Leu, Val, Phe, Tyr, Trp and Met
   X₃ is a hydrophobic amino acid, preferably selected from the group consisting of: Leu, Ile, Val, Phe, Tyr, Trp and Met
   X₅ is a hydrophobic amino acid selected from the group consisting of: Trp, Phe and Tyr
   X₂ and X₄ are anionic amino acids, preferably each independently is Asp or Glu
wherein the amino acids may be each independently a natural or non-natural amino acid, such as Cα or N methylated, peptoids, beta amino acids or D amino acids.

The present invention also provides a peptide comprising or consisting of the sequence from N-terminus to C-terminus:
X₋₂X₋₁ZX₁X₂X₃X₄X₅
wherein
Z is tyrosine, phosphotyrosine (pY) or a non-natural analogue of phosphotyrosine, such as phosphonodifluoromethyl phenylalanine (F₂Pmp)
X₋₂ is a hydrophobic amino acid, preferably selected from the group consisting of: Leu, Ile, Phe, Tyr, Trp and Met
X₋₁ is any amino acid
X₁ is a hydrophobic amino acid, preferably selected from the group consisting of: Ile, Val, Phe, Tyr, Trp and Met
X₃ is a hydrophobic amino acid, preferably selected from the group consisting of: Leu, Ile, Val, Phe, Tyr, Trp and Met
X₅ is a hydrophobic amino acid selected from the group consisting of: Trp, Phe and Tyr
X₂ and X₄ are anionic amino acids, preferably each independently is Asp or Glu wherein the amino acids may be each independently natural or non-natural amino acids, such as Cα or N methylated, peptoids, beta amino acids or D amino acids.

The present invention also provides a peptide comprising or consisting of the sequence from N-terminus to C-terminus:
X₋₂X₋₁ZX₁X₂X₃X₄X₅
wherein
   Z is a non-natural analogue of phosphotyrosine, such as phosphonodifluoromethyl phenylalanine (F₂Pmp), tyrosine or phosphotyrosine (pY)
   X₋₂ is a hydrophobic amino acid, preferably selected from the group consisting of: Leu, Ile, Val, Phe, Tyr, Trp and Met
   X₋₁ is any amino acid
   X₁ is a hydrophobic amino acid, preferably selected from the group consisting of: Ile, Leu, Val, Phe, Tyr, Trp and Met
   X₃ is a hydrophobic amino acid, preferably selected from the group consisting of: Leu, Ile, Val, Phe, Tyr, Trp and Met
   X₅ is Trp
   X₂ and X₄ are anionic amino acids, preferably each independently is Asp or Glu
wherein the amino acids may be each independently a natural or non-natural amino acid, such as Cα or N methylated, peptoids, beta amino acids or D amino acids.

In a further embodiment the invention provides a peptide comprising or consisting of the sequence from N-terminus to C-terminus:

X₋₂X₋₁ZX₁X₂X₃X₄X₅ (SEQ ID NO:1)

wherein
   Z is a non-natural analogue of phosphotyrosine, such as phosphonodifluoromethyl phenylalanine (F₂Pmp), tyrosine or phosphotyrosine (pY)
   X₋₂ is a hydrophobic amino acid, preferably selected from the group consisting of: Leu, Ile, Val, Phe, Tyr, Trp and Met
   X₋₁ is any amino acid
   X₁ is a hydrophobic amino acid, preferably selected from the group consisting of: Ile, Leu, Val, Phe, Tyr, Trp and Met
   X₃ is a hydrophobic amino acid, preferably selected from the group consisting of: Leu, Ile, Val, Phe, Tyr, Trp and Met
   X₅ is a hydrophobic amino acid, preferably selected from the group consisting of: Trp, Leu, Ile, Val, Phe, Tyr, and Met
   X₂ and X₄ are anionic amino acids, preferably each independently is Asp or Glu
wherein the amino acids may be each independently a natural or non-natural amino acid, such as Cα or N methylated, peptoids, beta amino acids or D amino acids, with the proviso that the peptide is not GLNpYIDLDL (SEQ ID NO:2) and GDKQVEpYLDLDLD (SEQ ID NO:3).

In a preferred embodiment of the peptides according to the invention:
X₋₂ is Leu and/or
X-1 is Asn and/or
X₁ is Ile and/or
X₂ is Asp and/or
X₃ is Leu and/or
X₄ is Asp and/or
X₅ is Trp, or Phe, preferably Trp.

Preferably Z is a non-natural analogue of phosphotyrosine, preferably Z is F₂Pmp. Preferably said analogue is a non-dephosphorylable derivative.

In a preferred embodiment of the invention, in any of the peptides as above defined the hydrophobic amino acid in X₅ is an aromatic amino acid, said aromatic amino acid being a natural or non natural amino acid, preferably tryptophane or phenyl alanine or 1-naphtyl-alanine or 2-naphtyl-alanine.

Preferably the peptide of the invention comprises or consists of the sequence from N-terminus to C-terminus:

LN-(F₂Pmp)-IDLDW (SEQ ID NO:4) or GLN-(F₂Pmp)-IDLDW (SEQ ID NO:5).

Preferably, the peptide of the invention comprises or consists of the peptides of Table 1 comprising or consisting of the sequence from N-terminus to C-terminus X₋₂X₋₁ZX₁X₂X₃X₄X₅ as indicated above.

In a preferred embodiment, the N-terminus and/or the C-terminus of the sequence or of the peptide as above defined are modified, preferably by acetylation or amidation, or by labelling with fluorescent probes, preferably 5, 6 carboxyfluorescein (CF) and Cyanine 3 carboxylic acid (Cy3), more preferably the N-terminus is acetylated or labeled with CF and/or the C-terminus is amidated.

Preferably, the peptide is the peptide comprising or consisting of the sequence from N-terminus to C-terminus:

LN-(F₂Pmp)-IDLDW or GLN-(F₂Pmp)-IDLDW (SEQ ID NO:5).

wherein the N-terminus of the peptide is acetylated, and the C-terminus of the peptide is amidated.

Preferably, the peptide according to the invention further comprises an aminoacidic sequence which favors penetration inside cells (cell-penetrating peptides), preferably said sequence is TAT (GRKKRRQRRR (SEQ ID NO: 25)), penetratin (RQIKIWFQNRRMKWKKGG(SEQ ID NO: 26)), transportan 10 (AGYLLGKINLKALAALAKKIL(SEQ ID NO: 11)) oligo-Arg (e.g. Arg₅ to Arg₁₂), said amino acidic sequence being linked to the N-terminus and/or the C-terminus of the peptide.

A further object of the invention is a non-covalent complex comprising the peptide as above defined and an aminoacidic sequence which favors penetration inside cells, preferably said sequence is Pep1 (Acetyl-KETWWETWWTEWSQPKKKRKV-Cysteamide (SEQ ID NO: 27)).

Another object of the invention is a pharmaceutical composition comprising a peptide as above defined or the complex as above defined and at least one pharmaceutically acceptable carrier, excipient and/or diluent, preferably further comprising at least one therapeutic agent, preferably said other therapeutic agent is selected from the group consisting of: chemotherapeutic agents, targeted anticancer agents, DNA damage response inhibitors.

Preferably the peptide as above defined, or the complex or the pharmaceutical composition as above defined as for use as a medicament, preferably for use:
in the treatment of childhood myeloproliferative disorders, preferably juvenile myelomonocytic leukemia (JMML), childhood myelodysplastic syndromes (e.g., RAEB), childhood leukemia (e.g., acute monocytic leukemia (AMoL, FAB M5) and acute lymphoblastic leukemia (ALL), “common” subtype), adult myelodysplastic syndromes, myelogenous and lymphoblastic leukemia, pediatric/adult solid tumors associated with an aberrant activity of SHP2 due to the occurrence of somatic PTPN11 gain-of-function mutations, e.g. neuroblastoma, glioma, embryonal rhabdomyosarcoma, lung cancer, colon cancer, and melanoma,
in the treatment of tumors associated with hyperactivation of the signal transduction pathways regulated by SHP2 (RAS-MAPK and PI3K-AKT-mTOR), e.g. colon, cervix, endometrium, pancreas, large and small intestine, skin, prostate, head and neck, and lung tumors,
in the treatment of post-natal clinical manifestations of RASopathies caused by germline mutations of PTPN11 (Noonan syndrome and Noonan syndrome with multiple lentigines, also called LEOPARD syndrome), such as hypertrophic cardiomyopathy, short stature and predisposition to certain malignancies, particularly JMML,
in cancer immunotherapy to avoid the tumor immune evasion mediated by SHP2’s activation of the immune checkpoint pathways, such as Programmed Cell Death 1 (PD-1) or signal-regulatory protein alpha (SIRPα)/CD47, thus modulating the immune response in cancer,
as a cytotoxin-associated gene A (CagA) competitor in H. pylori-mediated gastric carcinoma.

Another object of the invention is the use of the peptide as above defined, the complex or the pharmaceutical composition as above defined for investigating in vitro or ex vivo the role of SHP2’s interactions with its partners in the control of SHP2 function and the consequences of its dysregulation in SHP2-dependent signal transduction pathways.

The peptide according to the invention may e.g. comprise or have the sequence from N-terminus to C-terminus:
X₋₃X₋₂X₋₁pYX₁X₂X₃X₄X₅
wherein X-3 may be any amino acid, e.g. Gly, able to function as a spacer for linking other groups, such as fluorophores or cell-penetrating peptides;
wherein X₋₃ through X₅ have the same meaning as described above.

In the peptides according to the invention chemical groups, forming amide bonds, may be added to the N-terminal amino group or to the C-terminal carbonyl of the peptide.

E.g. a free amine may be present at the N-terminus or C-terminus; similarly, an acetyl group may be added at the N-terminus or C-terminus thus generating an N- or C-terminal acetylated peptide, respectively. Further peptides may be added at the N- or C-terminus of the peptides according to the invention, e.g. cell-penetrating peptides (CPPs), e.g. Pep1, TAT sequences, penetrantin, oligo-Arg, etc. Alternatively, such peptides may be combined with the peptides of the invention in different ratios, thus forming noncovalent complexes. Fluorescents labels may be added to the peptides of the invention, e.g. carboxyfluorescein or cyanine.

The invention relates also to a polynucleotide coding for the peptides as above defined, to a vector comprising the above polynucleotide and to a host cell genetically engineered which expresses the peptide as above defined. Preferably the polynucleotide is selected from the group consisting of: RNA or DNA, preferably said polynucleotide is DNA.

Preferably the vector is an expression vector selected from the group consisting of: plasmids, viral particles and phages.

Preferably said host cell is selected from the group consisting of: bacterial cell, fungal cell, insect cell, animal cell and plant cell, preferably said host cell is an animal cell.

The peptides of the invention may be in the form of synthetic or recombinant, linear and multimeric peptides in any chemical, physical and/or biological form such as to maintain their function.

The peptides of the invention may be synthetized and used in the branched form, as disclosed e.g. in the patent US 5,229,490.

The peptides of the invention may comprise two phosphorylated sequences for binding the two SH2 domains of SHP2, instead of only one of them.

All the amino acids in the peptide may have the same stereochemistry, for example, the peptide may consist of only L-amino acids or only D-amino acids. Alternatively, the peptide may comprise a combination of both L- and D- amino acids. The peptide of the present invention may be in the form of a dimer or multimer. In the present specification, examples of spacers comprised in the dimer or multimer include ester bonds (—CO—O—, —O—CO—), ether bonds (—O—), amide bonds (NHCO, CONH), sugar chain linkers, polyethylene glycol linkers, peptide linkers, and the like. Examples of peptide linkers include linkers containing at least one of 20 natural amino acids that constitute a protein. The number of amino acids of the peptide linker is, for example, but not limited to, 1 to 20, 1 to 15, 1 to 12, 1 to 10, 1 to 8, 1 to 6, or 1 to 4. Examples of peptide linkers include arginine dimer, arginine trimer, arginine tetramer, lysine dimer, lysine trimer, lysine tetramer, glycine dimer, glycine trimer, glycine tetramer, glycine pentamer, glycine hexamer, alanine-alanine-tyrosine (AAY), isoleucine-leucine-alanine (ILA), arginine-valine-lysine-arginine (RVKR), and the like, proline-alanine-serine (PAS) peptides. The spacer may be divalent or multivalent.

As used herein, the definition “hydrophobic amino acid” encompasses natural and non natural amino acids with non polar side chain. In particular, as used herein the term hydrophobic amino acid includes natural and non natural aromatic amino acids and natural and non natural aliphatic amino acids. The natural and non natural aromatic amino acids, also referred as aromatic amino acids, preferably comprise phenylalanine, tryptophan, tyrosine, phenylglycine, homophenylalanine, 2-methyl-phenylalanine, 3-methyl-phenylalanine, 4-methyl-phenylalanine, 3,4-dimethyl-phenylalanine, 4-tert-butyl-phenylalanine, 3-ethyl-phenylalanine, benzylcysteine, 3,3-diphenylalanine, 4,4-biphenylalanine, 1-naphtyl-alanine, 2-naphtyl-alanine, 3-(9-anthryl)-alanine, alpha-amino-2-indanacetic acid, 5-methyl-tryptophan, 6-methyl-tryptophan, 3-(2-quinolyl)alanine, 3-(3-quinolyl)alanine, 3-(4-quinolyl)alanine, 3-(5-quinolyl)alanine, 3-(6-quinolyl)alanine, 3-(2-quinoxalyl)alanine, styrylalanine. Even more preferably said aromatic amino acids are phenylalanine, tryptophan, tyrosine, 1-naphtyl-alanine and 2-naphtyl-alanine.

As indicated above, the term “hydrophobic amino acids” further refers to natural and non natural aliphatic amino acids with a non polar side chain. Said aliphatic amino acids comprise for example glycine, alanine, valine, leucine, isoleucine, proline, methionine, 2-aminobutyric acid, norvaline, homoleucine, beta-hydroxyleucine, norleucine, allo-isoleucine, tert-leucine, 2-aminoheptanoic acid, ethionine, cyclohexylglycine, 3-cyclohexyl-alanine, 3-cyclopentylalanine, adamanthane.

When the peptide of the present invention is a multimer, a branched multivalent linker (e.g., dendrimer), a metal complex, or the like may be used for linkage.

Examples of the branched multivalent linker include diethylenetriamine, spermine, spermidine, triethanolamine, ethylenediaminetetraacetate (EDTA), pentaerythritol, azido-propyl(alkyl)amine, lysine, omithine, asparagic acid, glutamic acid, polyfunctional peptides (lysine, omithine, or asparagic acid- or glutamic acid-containing dipeptide, tripeptide, or tetrapeptide), and organic multivalent amino compounds (e.g., poly(amidoamine) (PAMAM), tris(ethyleneamine)ammonia, and poly(propyleneimine) (Astramol (trademark))). The dendrimer of the present invention includes, for example, a dimer obtained by connecting the N-terminals (or C-terminals) of peptides herein disclosed. Further, the dendrimer of the present invention also includes a tetramer obtained by further connecting a “dimer obtained by connecting the N-terminals of the above peptides” and a “dimer obtained by connecting the C-terminals of the above peptides.”

Included in the present invention are also functional fragments, derivates or variants of the peptides above defined. Suitably, “derivatives” or “variants” include those in which instead of the naturally occurring amino acid the amino acid which appears in the sequence is a structural analog thereof. Amino acids used in the sequences may also be derivatized or modified, e.g. labelled, providing the function of the peptide is not significantly adversely affected. Derivatives and variants as described above may be prepared during synthesis of the peptide or by post-production modification, or when the peptide is in recombinant form using the known techniques of site- directed mutagenesis, random mutagenesis, or enzymatic cleavage and/or ligation of nucleic acids.

Functional “fragments” according to the invention may be made by truncation, e.g. by removal of one or more amino acids from the N- and/or C-terminal ends. Such fragments may be derived from the sequences herein disclosed or may be derived from a functionally equivalent peptide as described above.

Suitably, functional variants according to the invention have an amino acid sequence which has more than 70%, e.g. 75 or 80%, preferably more than 85%, e.g. more than 90 or 95% homology to the sequences herein disclosed.

Peptides of the invention, as defined herein, may be chemically modified, for example, post-translationally modified. For example, they may be glycosylated or comprise modified amino acid residues.

Chemically modified peptides also include those having one or more residues chemically derivatized by reaction of a functional side group. Such derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzyl histidine.

Also included as chemically modified peptides are cyclised peptides, i.e. peptides of the invention which are linked with a covalent bond to generate a ring. Typically an amino terminus and a carboxy terminus (so called head-to-tail cyclisation), an amino terminus and a sidechain (so called head-to-sidechain cyclisation), carboxy terminus and a sidechain (so called sidechain-to-tail cyclisation), or a side chain and a side chain (so called sidechain-to-sidechain cyclisation) may be linked with a covalent bond to form a cyclic peptide. Head-to-tail cyclic peptides may typically be formed by amide bond formation. Sidechain-to-sidechain cycles may typically be formed via Cys-Cys disulfide bridge formation or amide bond formation within a cyclic peptide. Alternatively, an amino terminus, a carboxy terminus or a side chain may be linked with a covalent bond to the peptide backbone to form a cyclic peptide.

Also included as chemically modified peptides are those which contain one or more naturally occurring amino acid derivatives of the twenty standard amino acids. For example, 4-hydroxyproline may be substituted for proline or homoserine may be substituted for serine.

A peptide of the invention may carry a revealing label. Suitable labels include radioisotopes, fluorescent labels, enzyme labels, or other protein labels such as biotin.

Any formula given herein is also intended to represent unlabeled forms as well as isotopically labeled forms of the peptides. Isotopically labeled peptides have structures depicted by the formulas given herein except that one or more atoms are replaced by an atom having a selected atomic mass or mass number. Examples of isotopes that can be incorporated into peptides of the invention include isotopes of hydrogen, carbon, nitrogen, oxygen, phosphorous, fluorine, and chlorine. Pharmaceutically acceptable solvates in accordance with the invention include those wherein the solvent of crystallization may be isotopically substituted, e.g. D2O, d6-acetone, d6-DMSO.

Peptides as described above for use in accordance with the invention may be prepared by conventional modes of synthesis including genetic or chemical means.

Synthetic techniques, such as a solid-phase Merrifield-type synthesis, may be preferred for reasons of purity, antigenic specificity, freedom from unwanted side products and ease of production. Suitable techniques for solid-phase peptide synthesis are well known to those skilled in the art (see for example, Merrifield et al., 1969, Adv. Enzymol 32, 221-96 and Fields et a/., 1990, Int. J. Peptide Protein Res, 35, 161-214). Chemical synthesis may be performed by methods well known in the art involving cyclic sets of reactions of selective deprotection of the functional groups of a terminal amino acid and coupling of selectively protected amino acid residues, followed finally by complete deprotection of all functional groups. Synthesis may be performed in solution or on a solid support using suitable solid phases known in the art.

In an alternative embodiment a peptide of the invention may be produced from or delivered in the form of a polynucleotide which encodes, and is capable of expressing, it. Such polynucleotides can be synthesised according to methods well known in the art, as described by way of example in Sambrook et al (1989, Molecular Cloning - a laboratory manual; Cold Spring Harbor Press). Such polynucleotides may be used in vitro or in vivo in the production of a peptide of the invention. Such polynucleotides may therefore be administered or used in the treatment of cancer or another disease or condition as described herein.

The present invention also includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al (ibid).

Thus, the peptide may be provided by delivering such a vector to a cell and allowing transcription from the vector to occur. Suitably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

The invention also includes cells that have been modified to express a peptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors encoding for a peptide of the invention include mammalian HEK293T, CHO, HeLa and COS cells. Suitably the cell line selected will be one which is not only stable, but also allows for mature glycosylation and cell surface expression of a polypeptide. Expression may be achieved in transformed oocytes. A suitable peptide may be expressed in cells of a transgenic non-human animal, in particular a mouse. A transgenic non-human animal expressing a peptide of the invention is included within the scope of the invention. A peptide of the invention may also be expressed in Xenopus laevis oocytes or melanophores.

The peptides of the present invention may be employed alone as a sole therapy or in combination with other therapeutic agents for the prevention and/or treatment of the above-mentioned conditions.

The peptide can be administered as separate compositions (simultaneous, sequential) of the individual components of the treatment or as a single dosage form containing both agents. When the peptides of this invention are in combination with others active ingredients, the active ingredients may be separately formulated into single-ingredient preparations of one of the above-described forms and then provided as combined preparations, which are given at the same time or different times, or may be formulated together into a two- or more- ingredient preparation.

As is common practice, the compositions are normally accompanied by written or printed instructions for use in the treatment in question.

The expert in the art will select the form of administration and effective dosages by selecting suitable diluents, adjuvants and / or excipients.

The present invention is therefore illustrated by means of non-limiting examples in reference to the following figures.

FIG. 1: SHP2 structure and scheme of the activation process.

Top: crystallographic structures for the closed, auto-inhibited and the open, active states of SHP2 (left and right, respectively). The N-SH2, C-SH2 and PTP domain are colored in light grey, black and white, respectively. The N-SH2 blocking loop (DE loop) and the PTP active site are highlighted in black. PDB codes of the two structures are 2SHP and 6CRF. Segments missing in the experimental structures were modeled as previously described [Bocchinfuso 2007].

Bottom: schematic model of the allosteric regulation mechanism.

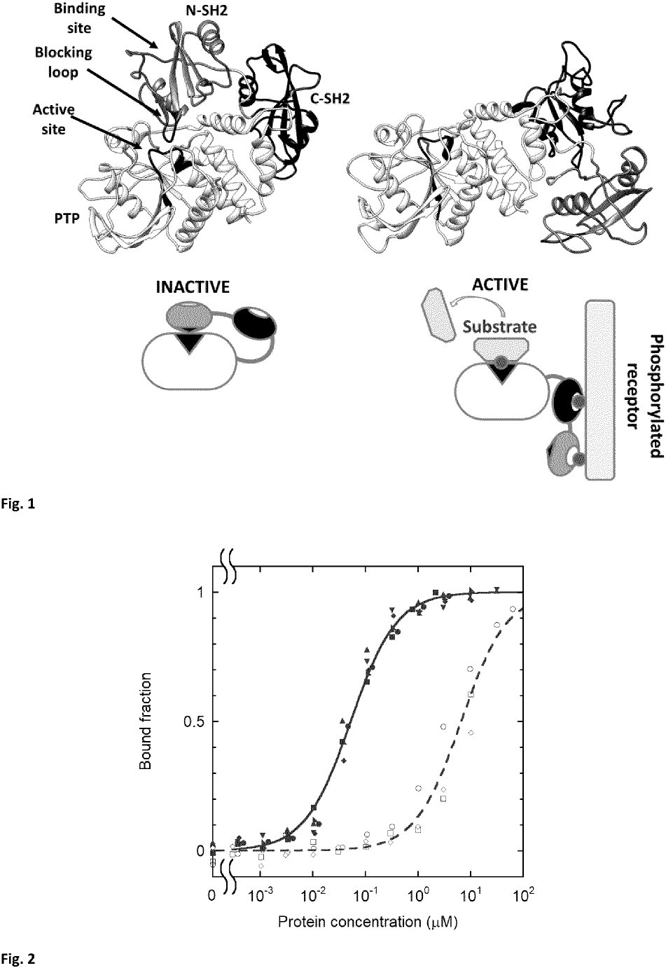

FIG. 2: Binding curves for the phosphorylated and unphosphorylated IRS-1 pY1172 peptides.

[CF-P9]=1.0 nM (full symbols and solid line), [CF-P9Y0]=10 nM (empty symbols and dashed line); Replicate experiments are reported with different symbols and were fit collectively.

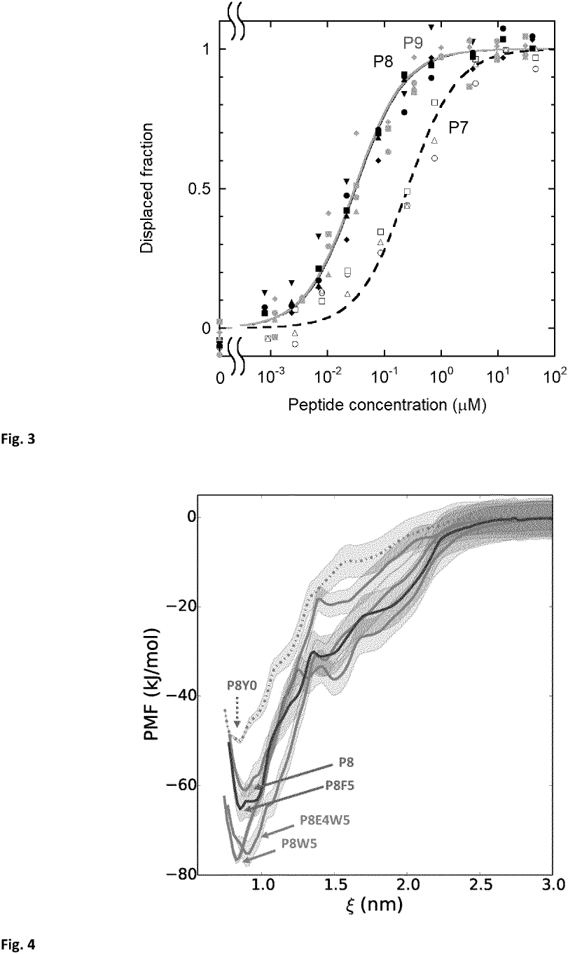

FIG. 3: effect of sequence length on the binding affinity.

Displacement experiments for analogs of different length. [CF-P9]=1.0 nM; [N-SH2]= 40 nM.

FIG. 4: in silico free energy calculations for different modified sequences

The free energy profile is reported as a function of the distance between the centers of mass of the N-SH2 domain and of the phosphopeptide. The simulations predict a loss in affinity of P8 with dephosphorylation of the pY (P8Y0), and a gain with substitution of the Leu in position +5 with Trp (P8W5), but not with Phe P8F). The additional substitution of Asp in +4 with Glu (P8E4W5) does not provide any further increase in affinity. Shaded areas correspond to standard deviations in the PMF profile.

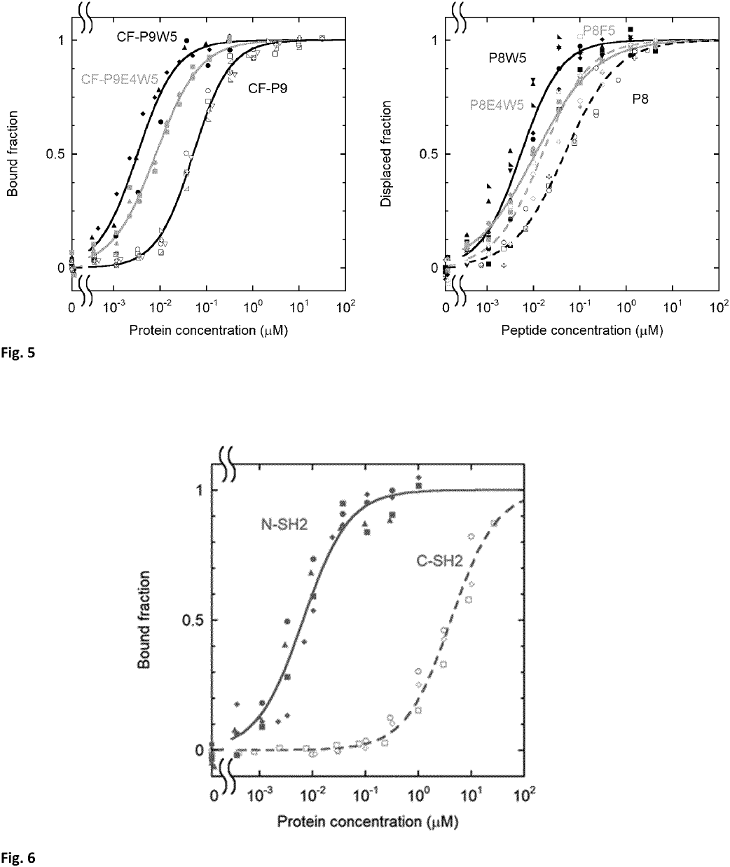

FIG. 5: effect of substitutions at position +5 on the binding affinity

Left panel: direct binding experiments; [CF-P9W5]=0.10 nM, [CF-P9E4W5]=0.10 nM, [CF-P9]=1.0 nM. Data for CF-P9 are repeated here for comparison.

Right panel: displacement assay, [CF-P9W5]=0.10 nM, [N-SH2]=3.35 Nm

FIG. 6: binding selectivity of CF-P9W5 for the two SH2 domains of SHP2

Comparison of the association curves of CF-P9W5 to the N-SH2 and C-SH2 domains of SHP2. Experimental conditions for the N-SH2 binding experiments: see FIG. 5; for the C-SH2 binding experiments: [CF-P9W5]=1.0 nM.

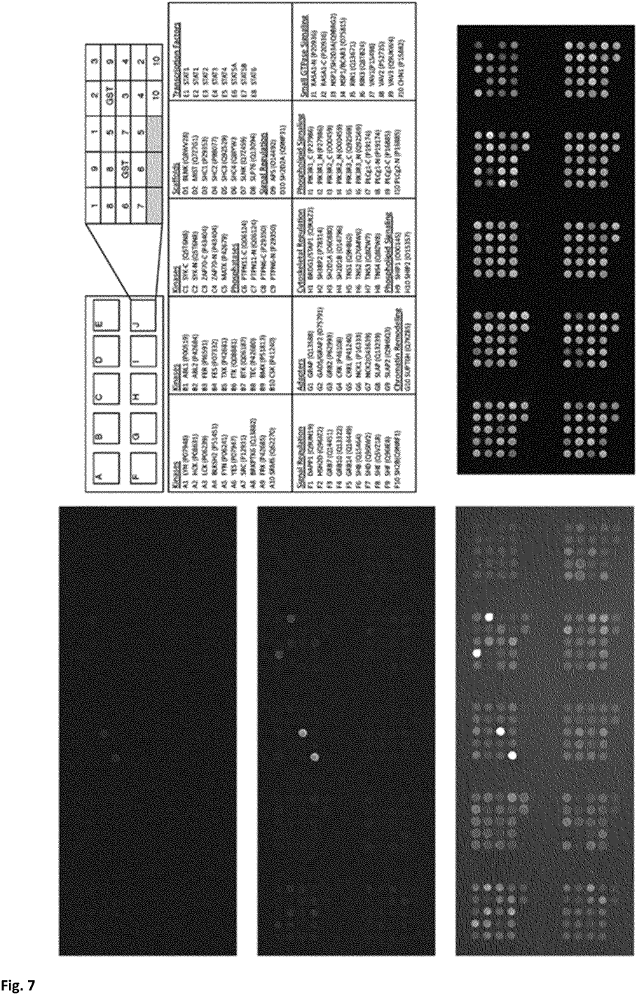

FIG. 7: binding selectivity of Cy3-P9W5 for an array of SH2 domains.

The left panels report the fluorescence of the bound peptide, at a concentration of 0.5 nM (top), 5.0 nM (center) and 50 nM (bottom). Each SH2 domain is present in duplicate, and negative control spots (with GST only) are also present. The bright spots correspond to the N-SH2 domain of SHP2 and to the SH2 domain of the SH2 and PH domain-containing adapter protein APS (also called SHP2B2). The intensity of all other spots is comparable to that of the negative controls.

The top right panel shows the position of each SH2 domain in the array, while the top bottom panel is a control of the protein loading in each spot, performed with an anti-GST antibody.

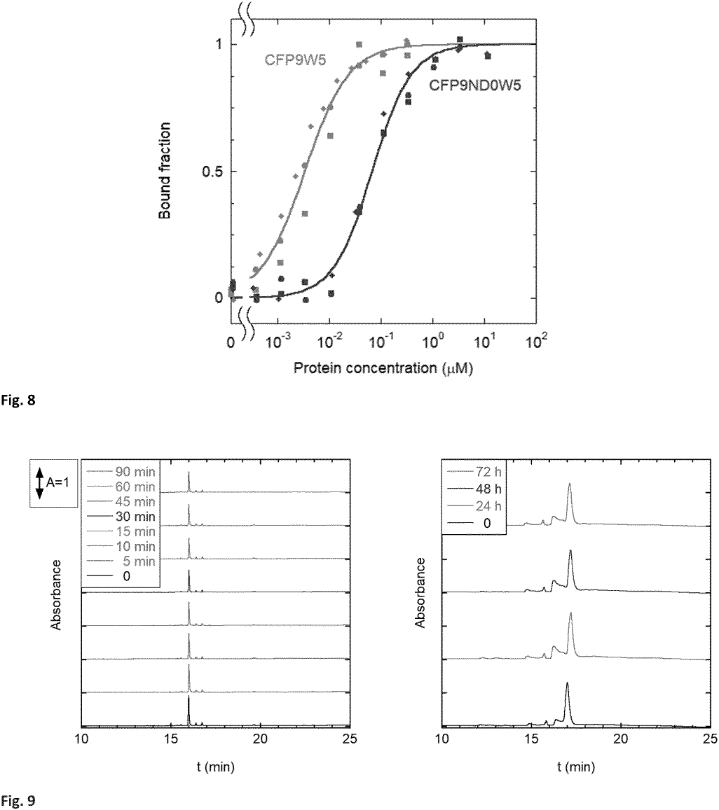

FIG. 8: binding of the non-dephosphorylatable peptide CF-P9ND0W5 (or OP) to the N-SH2 domain.

For comparison, the curve for CF-P9W5 is also shown. [CF-P9ND0W5]=1.0 nM, [CF-P9W5]=0.10 nM

FIG. 9. OP resistance to proteolytic degradation in human serum and in DMEM

HPLC profiles of OP, after incubation with human serum (left) or DMEM (right). Profiles are reported in order of increasing incubation time, from bottom to top.

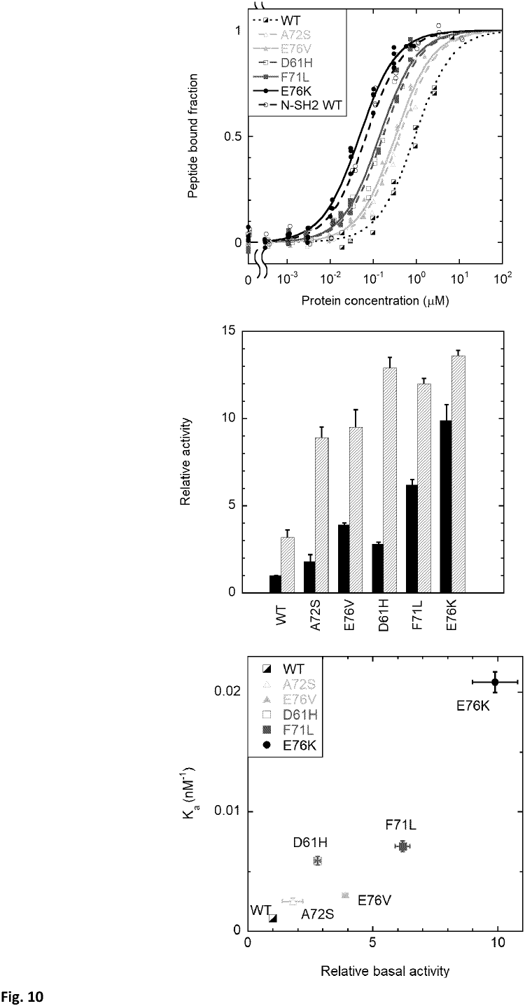

FIG. 10 Binding of the CF-OP peptide to the whole SHP2 protein (WT and pathogenic mutants)

Top panel: fraction of CF-OP peptide bound to the WT protein and selected mutants, obtained from fluorescence anisotropy experiments. The peptide bound fractions were obtained by following the variation of the peptide fluorescence anisotropy during the titration with increasing amounts of protein. [CF-OP]=1.0 nM.

Center panel: catalytic activity of the WT protein and selected mutants, under basal conditions (black bars) and after stimulation with 10 µM BTAM peptide (grey bars).

Bottom panel: correlation between basal activity and affinity (association constant).

FIG. 11: dephosphorylation of P8W5 and other phosphopeptides by the SHP2 PTP domain.

The following phosphopeptides were used for comparison, in addition to P8 and P8W5, in a malachite green assay.

GAB1 Y657 (DKQVEpYLDLDL (SEQ ID NO:6))

p190A/RhoGAP Y1105 (EEENIpYSVPHD(SEQ ID NO:7))

EGFR Y1016 (VDADEpYLIPQQ(SEQ ID NO:8))

BTAM, or biphosphorylated SHSP-1 TAM1

(GGGGDIT(pY)ADLNLPKGKKPAPQAAEPNNHTE(pY)ASIQTS (SEQ ID NO:9),

with 4 N-terminal G residues)

A SHP2 construct lacking the N-SH2 domain (i.e. the first 104 residues) was used at a 95 nM concentration. Phosphopeptides were added at a 100 µM concentration and the phosphate released was measured at different times. From the linear region of the phosphate versus time curve, the variation in absorbance at 655 nm in 1 min, due to phosphate released, was calculated and plotted.

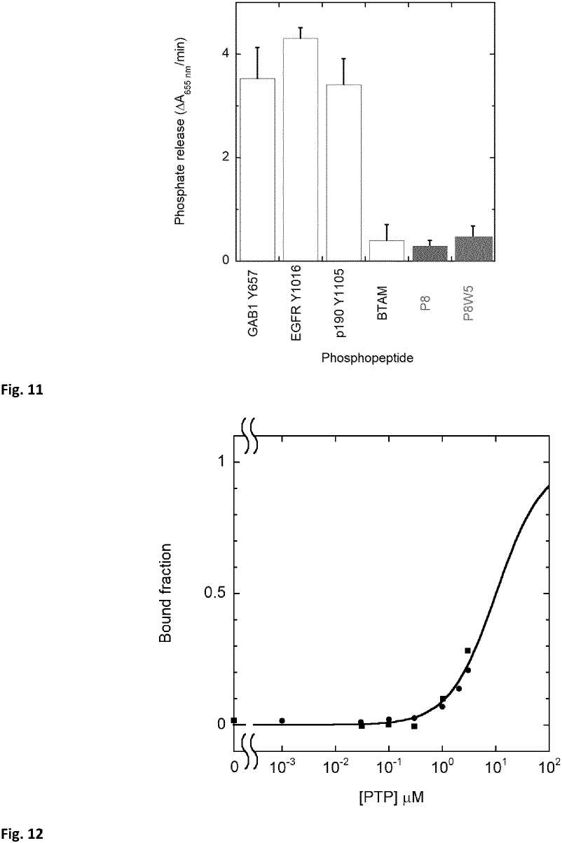

FIG. 12. CF-OP association to the PTP domain.

[CF-P9ND0W5]=1.0 nM

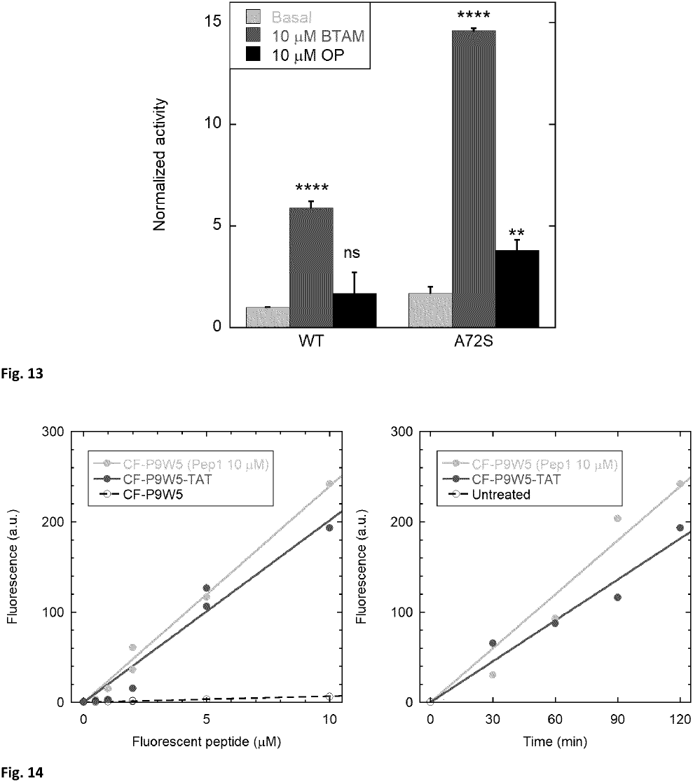

FIG. 13: SHP2 activation by the OP

Basal activity is reported in light grey, while activities in the presence of 10 µM BTAM or 10 µM OP are shown in dark grey and black, respectively.

FIG. 14: cell uptake of the phosphopeptides

Left panel: FACS quantification of spontaneous and CPP-induced cell uptake in NIH3T3 cells after 120 minutes of incubation.

Right panel: time course of the cell uptake process, at a 10 µM fluorescent peptide concentration.

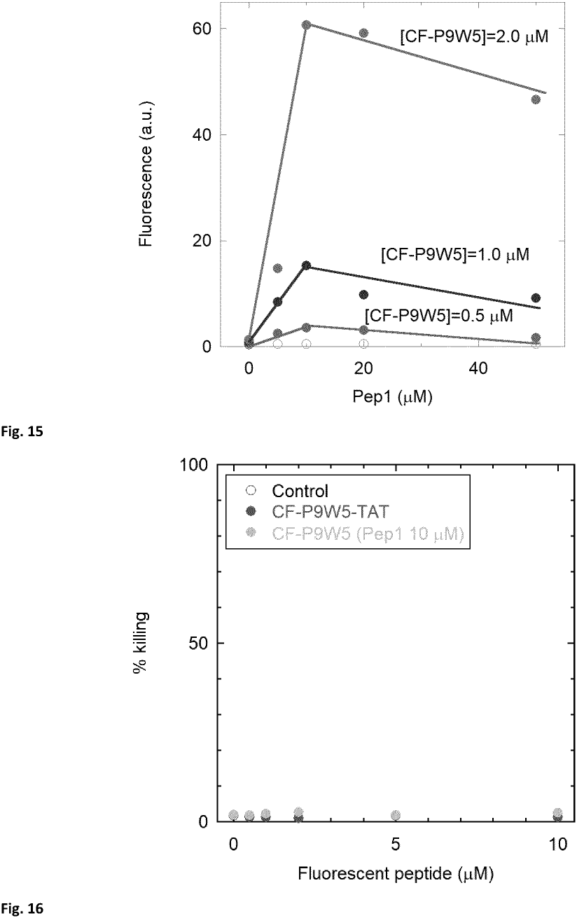

FIG. 15: optimization of Pep1 concentration

FACS quantification of Pep1-induced cell uptake in NIH3T3 cells after 120 minutes of incubation. CF-P9W5 concentration was 0, 0.5 µM, 1.0 µM or 2.0 µM.

FIG. 16: peptide toxicity in the cell uptake experiments

Cell viability in the FACS experiment was determined by using Sytox fluorescence.

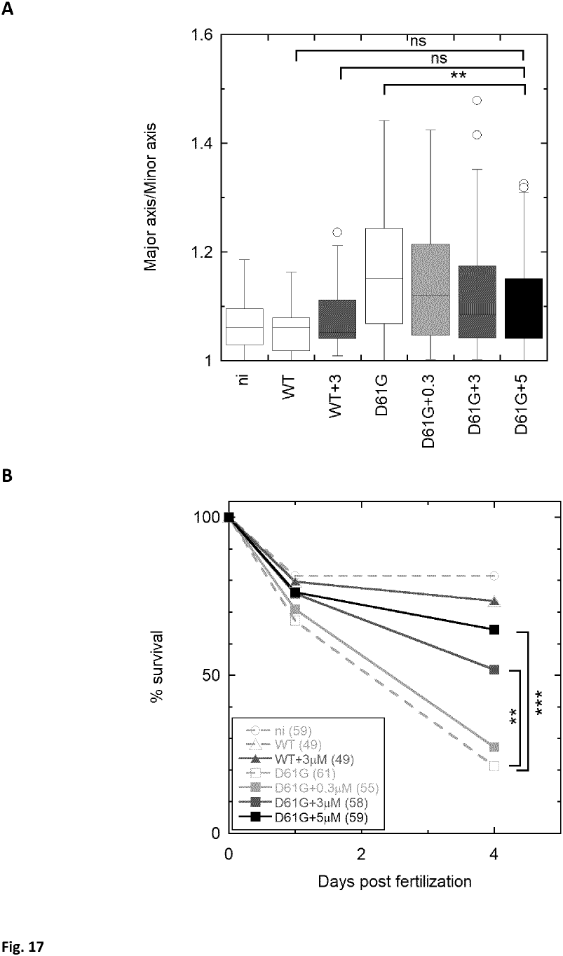

FIG. 17: the OP partially rescues Shp2a-D61G-induced gastrulation defects and mortality in a dose dependent manner in zebrafish embryos. Embryos were injected at the one-cell stage with mRNA encoding GFP-2A-Shp2-D61G or GFP465 Shp2-wt with or without peptide at 0.3 µM, 3 µM and 5 µM concentration. Non-injected embryos (ni) were evaluated as a control. A: ovality of embryos at 11 hpf, as indicated by the ratio of the long and the short axis. Tukey’s honest significant difference test was done to assess significance. In the box plot, the horizontal line indicates the median, box limits indicate the 25th and 75th percentiles (interquartile range), whiskers (error bars) extend to the maximum and minimum values, or to 1.5 times the interquartile range from the 25th and 75th percentiles, if some data points fall outside this range. In the latter case, outliers are indicated as single data points. B: embryo lethality. Surviving embryos were counted at 1 dpf and 4 dpf. Survival was plotted and Log-rank test was done to access differences between groups. Non significant (ns) p > 0.05; * p < 0.05; ** p < 0.01; *** p < 0.001. The number of embryos that were analyzed are indicated in parentheses.

### EXAMPLES

### Materials and Methods

### Materials and Methods

### Materials

Fmoc fluorenylmethyloxycarbonyl)-amino acids were obtained from Novabiochem (Merck Biosciences, La Jolla, CA). Rink amide MBHA resin (0.65 mmol/g, 100-200 mesh) was purchased from Novabiochem. All other protected amino acids, reagents and solvents for peptide synthesis were supplied by Sigma-Aldrich (St. Louis, MO). The LB medium components, all the reagents used to prepare the buffers and the Bradford reagent were purchased from Sigma Aldrich. tris(2-carboxyethyl)phosphine (TCEP) was obtained from Soltec Ventures, Beverly, MA, USA. Spectroscopic grade organic solvents were purchased from Carlo Erba Reagenti (Milano, Italy). Cell culture media growth factors and antibodies were purchased from VWR International PBI (Milan, Italy), EuroClone (Milan, Italy), Promega (Madison, WI, USA), Invitrogen (Carlsbad, CA, USA), Cell Signaling (Danvers, MA, USA), Sigma-Aldrich (Saint Louis, MO, USA), and Santa Cruz Biotechnology (Dallas, TX, USA).

### Peptide Synthesis

Assembly of peptides on the Syro Wave (Biotage, Uppsale, Sweden) peptide synthesizer was carried out on a 0.1 mmol scale by the FastMoc methodology, beginning with the Rink Amide MBHA resin (Merck Biosciences, La Jolla, CA) (155 mg, loading 0.65 mmol/g). The peptide was cleaved from the resin, filtered and collected. The solution was concentrated under a flow of nitrogen, and the crude peptide precipitated by addiction on diethyl ether. The crude peptides were purified by flash chromatography on Isolera Prime chromatographer (Biotage, Uppsale, Sweden) using a SNAP Cartridge KP-C18-HS 12 g or preparative RP-HPLC on a Phenomenex C18 column (22.1x250 mm, 10 µm, 300 Å) using an Akta Pure GE Healthcare (Little Chalfont, UK) LC system equipped with an UV-detector (flow rate 15 mL/min) and a binary elution system: A, H2O; B, CH3CN/H2O (9: 1 v/v); gradient 25-55% B in 30 min. The purified fractions were characterized by analytical HPLC-MS on a Phenomenex Kinetex XB-C18 column(4.6 x 100 mm, 3.5 µm, 100 Å) with an Agilent Technologies (Santa Clara, CA) 1260 Infinity II HPLC system and a 6130 quadrupole LC/MS. The binary elution system used was as follows: A, 0.05% TFA (trifluoroacetic acid) in H2O; B, 0.05% TFA in CH3CN; flow rate 1 mL/min. Retention times (Rt) for the synthetic peptides obtained from RP-HPLC (the elution conditions used for different peptides are listed in brackets) and molecular weights for the synthetic peptides experimentally determined by ESI-MS spectrometry are reported in Table 1.

Peptides were dissolved in DMSO to obtain stock solutions between 1 and 1.5 mM. The exact concentration was obtained by UV measurements, exploiting the signal of carboxyfluorescein for the labeled peptides and of pTyr, Tyr and Trp for the unlabeled peptides. To this end, CF-labeled peptides were diluted from the stocks (1:100) in buffer (pH 9), and their concentration was calculated from the CF signal at 490 nm using a molar extinction coefficient of 78000 M-1 cm-1 [Esbjörner 2007]. Unlabeled peptides were diluted 1:100 in a pH 7.4 buffer; molar extinction coefficients of Tyr, Phe and Trp were taken from Pace et al. [1995], while molar extinction coefficient of pY was taken from Bradshaw et al. [1999].

### Protein Expression and Purification

The human esaHis-tagged PTPN11 (residues 1-528) cDNA was cloned in a pET-26b vector (Novagen, MA, USA). Nucleotide substitutions associated with NS or leukemia were introduced by site-directed mutagenesis (QuikChange site-directed mutagenesis kit; Stratagene, CA, USA). A construct containing the cDNA encoding the isolated PTP domain preceded by the C-SH2 domain (residues 105-528) was generated by PCR amplification of the full-length wild-type cDNA and subcloned into the pET-26b vector (SHP2Δ104). A similar procedure was followed for the constructs of the N-SH2 (residues 2-111), C-SH2 (109-217) and PTP (212-528) domains, and of the N-SH2/C-SH2 tandem (2-217). Primer sequences are available upon request.

Recombinant proteins were expressed as previously described [Martinelli 2012, Pannone 2017], using E.coli (DE3) Rosetta2 competent cells (Novagen). Briefly, following isopropil-β-D-1-tiogalattopiranoside (Roche) induction (2 hr at 30° C., or overnight at 18° C.), bacteria were centrifuged at 5,000 rpm, 4° C. for 15 minuts, resuspended in a lysozyme-containing lysis buffer (TRIS-HCl 50 mM , pH=8.5, NaCl 0.5 M, imidazole 20 mM, tris(2-carboxyethyl)phosphine (TCEP) ImM, lysozyme 100 mg/ml, 1 tablet of complete protease inhibition cocktail) and sonicated. The lysate was centrifuged at 16,000 rpm, 4° C. for 30 minutes. The supernatant was collected and the protein of interest was purified by affinity chromatography on a Ni-NTA column (Qiagen, Hilden, Germany), using a TRIS-HCl 50 mM, NaCl 0.5 M, TCEP 1 mM buffer containing 100 mM or 250 mM imidazole, for washing and elution, respectively.

To remove imidazole, the samples were then dialyzed in a 20 mM TRIS-HCl (pH 8.5) buffer, containing 1 mM TCEP and 1 mM EDTA and 50 mM NaCl (or 150 mM NaCl if no further purification steps followed). Full length proteins and the SHP2Δ104 construct were then further purified by sequential chromatography, using an Äkta FPLC system (Äkta Purifier 900, Amersham Pharmacia Biotech, Little Chanfont, UK). The samples were first eluted within an anion exchange Hi-Trap QP 1 ml-column (GE Helathcare, Pittsburgh, PA, USA); the elution was carried out using TRIS-HCl 20 mM (pH 8.5) in a NaCl gradient from 50 to 500 mM. The most concentrated fractions were then eluted in a gel filtration Superose column using TRIS-HCl 20 mM buffer containing NaCl (150 mM) as mobile phase . Sample purity was checked by SDS PAGE with Coomassie Blue staining and resulted to be always above 90%.

Proteins were quantitated by both the Bradford assay [Bradford 1976] and the UV absorbance of aromatic residues, calculating extinction coefficients according to Pace. [Pace 1995]. In general, the two methods were in agreement, but the values derived from UV absorbance were more precise and are reported in the Figures and Tables. The protein samples were used immediately after purification or stored at -20° C. and used within the following week. In this case, after thawing TCEP 2.5 mM was added, the samples were centrifuged at 13,000 rpm for 20 minutes, and the new concentration was re-evaluated. In the few cases where residual apparent absorbance due to light scattering was present in the UV spectra, it was subtracted according to Castanho et al., 1997.

### Phosphatase Activity Assays

Catalytic activity was evaluated in vitro using 20 pmol of purified recombinant proteins in a 200-uL reaction buffer supplemented with 20 mM p-nitrophenyl phosphate (Sigma) as substrate, either basally or following stimulation with the protein tyrosine phosphatase nonreceptor type substrate 1 (PTPNS1) bisphosphotyrosyl-containing motif (BTAM peptide) (GGGGDIT(pY)ADLNLPKGKKPAPQAAEPNNHTE(pY)ASIQTS(SEQ ID NO:9)) (Primm, Milan, Italy), as previously described [Martinelli 2008]. Proteins were incubated for 15 min (SHP2Δ104) or 30 min (SHP2) at 30° C. Phosphate release was determined by measuring absorbance at 405 nm.

DiFMUP (6,8-difluoro-4-methylumbelliferyl phosphate) assay was carried out as previously described [Garcia Fortanet 2016], with minor changes. Briefly, reactions were performed at room temperature in 96-well flat bottom, low flange, non-binding surface, black polystyrene plates (Corning, cat. no. 3991), using a final volume of 100 µl and the following assay buffer: 60 mM HEPES, pH 7.2, 75 mM NaCl, 75 mM KCl, 1 mM EDTA, 0.05% Tween-20, 5 mM DTT. Catalytic activity was checked using 1 nM SHP2 and different concentrations of activating peptides. After 45 min at 25° C., 200 µM of surrogate substrate DiFMUP (Invitrogen, cat. no. D6567) was added to the mix, and incubated at 25° C. for 30 min. The reaction was stopped by addition of 20 µl of 160 µM bpV(Phen) (Potassium bisperoxo(1,10-phenanthroline)oxovanadate (V) hydrate) (Enzo Life Sciences cat. no. SML0889-25MG). The fluorescence was monitored using a microplate reader (Envision, Perki-Elmer) using excitation and emission wavelengths of 340 nm and 455 nm, respectively.

The ability of SHP2 to dephosphorylate the phosphopeptides was evaluated through a malachite green phosphatase assay (PTP assay kit 1 Millipore, MA, USA). The BTAM peptide and the following monophosphorylated peptides derived from known SHP2 substrates were used for comparison: DKQVEpYLDLDL (SEQ ID NO:6) (GAB1Y657), EEENIpYSVPHD (SEQ ID NO:7) (p190A/RhoGAPY1105), and VDADEpYLIPQQ (SEQ ID NO:8) (EGFRY1016) (Primm) [Sun et al., 2009; Ren et al., 2011]. SHP2Δ104 or PTP (2.38 pmol) were incubated with 100 µM of each phosphopeptide (total volume 25 µl) for different times. The reaction was stopped by adding 100 µl of malachite green solution. After 15 min, absorbance was read at 655 nm using a microplate reader, and compared with a phosphate standard curve to determine the release of phosphate. Data obtained in the linear region of the curve were normalized on the reaction time (1 min).

### Fluorescence Anisotropy Binding Assay

Anisotropy measurements were carried out using a Horiba Fluoromax 4 spectrofluorimeter.

For the binding assays, the requested peptide amount (1 nM or 0.1 nM) was diluted in buffer (HEPES 10 mM, NaCl 150 mM, EDTA 1 mM, TCEP 1 mM, fluorescence buffer henceforth) and its anisotropy signal was recorded. The peptide was then titrated with increasing protein amounts, until the anisotropy signal reached a plateau at its maximum value, or up to a protein concentration where protein aggregation and consequent light scattering affected the anisotropy values (usually above 1 µM). The measurements of CF-labeled peptides were carried out using an excitation wavelength of 470 nm and collecting the anisotropy values at an emission wavelength of 520 nm. A 495 nm emission filter was used. For the Cy3-labeled peptides, excitation and emission wavelengths of 520 and 560 nm were used. The lowest peptide concentration needed to have a sufficient fluorescent signal (0.1 nM) was used in the binding experiments. Higher concentrations (1 or 10 nM) were used for peptides with lower affinities, and therefore higher K_{d} values.

The displacement assays were carried out with the same experimental settings. In this case, the labeled peptide-protein complex was titrated with increasing amounts of the unlabeled peptide, following the decrease in anisotropy. Measurements were carried out at the same CF-peptide concentration used for the corresponding binding experiments. Regarding the protein concentration, a compromise between two requirements is needed [Huang 2003]. On one hand, it is desirable to have a significant fraction of the CF-peptide bound to the protein, to maximize the dynamic range in the anisotropy signal, which decreases during the displacement experiment.

On the other hand, the protein concentration should be comparable or lower than the dissociation constant of the unlabeled peptide (Kᵢ), to allow a quantitative and reliable determination of its binding affinity. Since several unlabeled peptides had a higher affinity than their fluorescent counterparts, in the displacement assays authors used a protein concentration [P]_{T}~K_{d}, or in some cases even ~K_{d}/2.

The K_{d} values were obtained fitting the data with the following equation, described in Van der Weert 2011, which avoids the need for the commonly used (but often unjustified) approximation of the concentration of unbound protein with the total concentration:

$$\frac{r - {r}_{0}}{{r}_{m a x} - {r}_{0}} = \frac{{\left[P\right]}_{T} + {\left[L\right]}_{T} + {K}_{d} - \sqrt{{\left({\left[P\right]}_{T} + {\left[L\right]}_{T} + {K}_{d}\right)}^{2} - 4 {\left[P\right]}_{T} {\left[L\right]}_{T}}}{2 {\left[L\right]}_{T}}$$

Here, [P]_{T} and [L]_{T} are the total protein and ligand concentrations, while r, ro and rₘₐₓ are the anisotropy values at a given protein concentration, in the absence of protein and when the peptide is completely bound, respectively.

The affinity of unlabeled peptides was determined by competition experiments, in which a sample with fixed total protein and ligand concentrations ([P]_{T} and [L]_{T}) was titrated with the inhibitor, causing displacement of the fluorescent peptide and a decrease in anisotropy. From these data, the IC₅₀ (i.e. the total concentration of unlabeled peptide that displaces half of the bound fluorescent analog) was determined, interpolating the displacement curve using a phenomenological Hill equation [Barlow 1989]:

$$\frac{r - {r}_{0}}{{r}_{f i n} - {r}_{0}} = \frac{{\left\{\frac{{\left[I\right]}_{T}}{I {C}_{50}}\right\}}^{n}}{1 + {\left\{\frac{{\left[I\right]}_{T}}{I {C}_{50}}\right\}}^{n}}$$

where [I]_{T} is the total concentration of the peptide causing the displacement, and r_{fin} is the anisotropy corresponding to total displacement, while in this case r₀ is the starting anisotropy, in the absence of displacing peptide.

Successively, the dissociation constant of the unlabeled peptide (Kᵢ) was calculated from the know values of IC₅₀, K_{d}, [P]_{T} and [L]_{T}, as described here below.

$${K}_{i} = \frac{I {C}_{50} - {\left[P\right]}_{T} + \frac{{\left[P L\right]}_{0}}{2} \left(1 + \frac{{K}_{d}}{{\left[L\right]}_{T} - \frac{{\left[P L\right]}_{0}}{2}}\right)}{\left(\frac{2 {\left[P\right]}_{T}}{{\left[P L\right]}_{0}} - 1\right) \frac{{\left[L\right]}_{T} - \frac{{\left[P L\right]}_{0}}{2}}{{K}_{d}} - 1}$$

Here, [PL]₀ can be substituted with the following expression, analogous to Eq. (1):

$${\left[P L\right]}_{0} = \frac{{\left[P\right]}_{T} + {\left[L\right]}_{T} + {K}_{d} - \sqrt{{\left({\left[P\right]}_{T} + {\left[L\right]}_{T} + {K}_{d}\right)}^{2} - 4 {\left[P\right]}_{T} {\left[L\right]}_{T}}}{2}$$

In this way, Kᵢ is expressed as a function of the known quantities IC₅₀, K_{d}, [P]_{T} and [L]_{T}, without any approximation.

### SH2 Domain Microarray

The microarray experiment was conducted by the Protein Array and Analysis Core at the MD Anderson Cancer Center (University of Texas, USA), as previously described [Roth 2019]. Briefly, a library of SH2 domains [Huang 2008] was expressed as GST fusion in E. coli and purified on glutathione-sepharose beads. The domains were spotted onto nitrocellulose-coated glass slides (Oncyte Avid slides, Grace Bio-Labs) using a pin arrayer [Espejo 2002]. Each domain was spotted in duplicate. After incubation with a Cy3-P9W5 solution (0.5, 5.0 nM, or 50 nM), fluorescence signals were detected using a GeneTACTM LSIV scanner (Genomic Solutions).

### In Silico Studies

### System Preparation

The initial structure of the N—SH2 complexed with phosphopeptide P8 (Table 1) was obtained by amino acid substitutions (and deletion) in the crystallographic structure of the protein complexed with the GAB1 peptide (sequence GDKQVE-pY-LDLDLD (SEQ ID NO:3)) (PDB code 4QSY). Side-chain configurations for mutated residues were chosen as the most probable in a backbone-dependent rotamer library [Dunbrack 2002]. The obtained complex was then used as the starting structure for subsequent amino acid substitutions in the bound peptide.

### System Equilibration

MD simulations were performed using the GROMACS simulation package [Abraham 2015] and a variant of AMBER99SB force field with parameters for phosphorylated residues [Homeyer 2006]. Water molecules were described by the TIP3P model [Jorgersen 1983]. All the simulated systems were inserted in a pre-equilibrated triclinic periodic box containing about 24000 water molecules and counterions to neutralize system total charge. They were relaxed first by doing a minimization with 5000 steepest descent cycles, by keeping protein positions fixed and allowing water and ions to adjust freely, followed by a heating protocol in which temperature was progressively increased from 100 K to 300 K. The system was then equilibrated for 100 ps in the NVT ensemble at 300 K, using velocity rescaling with a stochastic term (relaxation time 1 ps) [Bussi 2007] and then for 500 ps at constant pressure (1 atm) using the Parrinello-Rhaman barostat (relaxation time 5 ps) [Parrinello 1981]. Long-range electrostatic interactions were calculated using the particle mesh Ewald method [Darden 1993] and the cut-off distance for the non-bonded interaction was set equal to 12.0 Å. The LINCS constraint to all the hydrogen atoms and a 2 fs time-step were used [Hess 1997].

### Sampling of the Initial Configurations for Umbrella Sampling

For each system, a set of initial configurations was prepared by performing a center-of-mass (COM) pulling simulation. The distance between the peptide and N-SH2 domain COMs was constrained with a harmonic force (K=1000 kJ mol-1 nm-2). Pulling was performed by gradually increasing the value of the equilibrium distance with a constant-rate of 0.0025 nm/ps. The length of each simulation was about 2.5 ns. During the whole simulation, a positional restraint (1000 kJ/(mol · nm) was applied to all heavy atoms in the N-SH2 domain except for atoms in loops around the binding region (residues 30-45, 52-75, 80-100). The choice of the optimal unbinding pathway is critical for a reliable estimation of the peptide binding free energy [Chovancova 2012, Vuong 2015]. In this work, three different directions were tested, corresponding to: i) the vector from the phosphate to the alpha carbon in pY, in the equilibrated complex; ii) the vector defined by the initial positions of the two COMs; iii) the vector perpendicular to the surface of the cavity flanked by the EF and BG loops, passing through the N-SH2 domain center of mass. Among the three different pathways, the third direction encountered less steric occlusion by the EF and BG loops, and was thus selected for further analyses.

### Umbrella Sampling Simulations

A set of starting configurations was extracted from the pull-dynamics trajectory saving the peptide-protein center-of-mass distances every 2 Å in the range from 9 to about 40 Å, thus obtaining about 20 windows along the COM distance. The system in each window was preliminarily equilibrated for 1 ns with a strong positional restraint (1000 kJ/(mol · nm) to all carbon alpha atoms except for those in loops flanking the binding region (as in the pull simulation), followed by a production run of 150 ns with the restraints. During this stage, an harmonic potential (K=1000 kJ/mol · nm2 ) was applied on the distance between the two COMs. Additional sampling windows were added every 1 Å along the distance between the two COMs up to a distance of 15 Å. The resulting asymmetric distribution of sampling windows was used to calculate PMF on the production run trajectories. The Weighted Histogram Analysis Method (WHAM) [Kumar 1992] was used, with default settings (50 bins and tolerance of 10-6 kJ mol-1), using the gmx wham GROMACS tool [Hub 2010]. The analysis of the simulation was carried out on the 150 ns production dynamics, during which configurations were stored every 0.1 ns. The statistical uncertainty of the obtained PMF was estimated by bootstrapping analysis [Hub 2010].

### Peptide Stability in Serum and in DMEM

The peptides were dissolved in DMSO (C=5 mg/mL). In eppendorf tubes, 1 mL of HEPES buffer (C = 25 mM, pH = 7.6) was temperature equilibrated at 37° C. before adding 250 µL of human serum and 20 µL of peptide solution; the reaction was followed for 90 minutes. At fixed intervals, 100 µL of the solution were withdrawn and added to 200 µL of absolute ethanol. These samples were kept on ice for 15 minutes, then centrifuged at 13,000 rpm for 5 minutes; the supernatant solutions were analyzed by HPLC and HPLC-MS with 20-60% B gradient in 20 minutes to follow the reaction. In parallel, samples containing peptide, buffer and ethanol only were also analyzed, too. A degradation resistance test was also conducted in DMEM (Dulbecco’s Modified Eagle Medium). The experimental conditions are similar to those described above; the reaction was followed for 72 hours. The enzymatic degradation resistance tests were followed by HPLC using a 5-50% B gradient in 20 minutes.

### Cell Uptake Experiments

Cell uptake experiments were performed on NIH3T3 cells. About 200,000 cells were seeded on six-well plates and cultured in Dulbecco’s modified Eagle’s medium supplemented with 10% heat-inactivated fetal bovine serum (Gibco) and 1 % penicillin-streptomycin, at 37° C. with 5% CO₂.

Two different strategies were followed. In a first set of experiments, the fluorescent peptide has been incubated with the cell-penetrating peptide (CPP) Pep-1. A Pep-1 stock solution was prepared by dissolving the peptide in ultrapure water. The fluorescent peptide was diluted in PBS from the DMSO stock solution. The peptides were mixed and incubated for 30 minutes at room temperature in a final volume of 400 µl. The conditions for cell delivery were optimized by investigating different cargo to CPP ratios. The mixture was then added to the cells, washed with PBS, together with 600 µl of serum-free medium (volumes for one well are reported). After one hour, 10% of fetal bovine serum (FBS) was added.

Cells were incubated for different times and then washed, collected and stained with SYTOX blue (1 mM, Invitrogen™) to identify the viable population and the green fluorescence of CFP9W5 was recordered to measure peptide uptake. Data were analysed with Kaluza Analysis Software (Beckman Coulter).

A second set of measurement was carried out using the TAT-conjugated peptide analog. In this case, cells were directly incubated with different peptide amounts.

### In Vivo Zebrafish Rescue Experiments

One cell stage zebrafish embryos were injected with a mixture of 120 ng/µl of mRNA encoding either GFP-2A-Shp2-D61G or GFP-2A-Shp2-wt (as a control), with or without OP, at 0.3 µM, 3 µM and 5 µM concentration. Embryos were selected based on proper GFP expression and imaged at 11 hours post fertilization (hpf) in their lateral position using the Leica M165 FC stereomicroscope. Images were analyzed using ImageJ [Schneider 2012], by measuring the ratio of the major and minor axis from a minimum of 31 embryos. Statistical analysis was performed in GraphPad Prism, using the analysis of variance (ANOVA) complemented by Tukey’s honest significant difference test (Tukey’s HSD). To measure the survival of injected embryos, a minimum of 48 embryos per group were grown up to 4 days post fertilization (dpf) and counted at 1 dpf and 4 dpf. Survival curves were plotted using GraphPad Prism, and the differences between samples were determined using the Log-rank (Mantel-Cox) test.

### Results

### 1) Characterization of IRS-1 pY1172/N-SH2 Binding

### 1.1) The IRS-1 pY1172 Peptide Binds the N-SH2 Domain With a Low Nanomolar Affinity

In a direct binding experiment, the fluorescently labeled peptide IRS-1 pY1172 analog CFP9 (Table 1) was titrated with increasing concentrations of the N-SH2 domain. The fraction of protein-bound peptide was determined from the increase in fluorescence anisotropy (FIG. 2), and a KD of 53±2 nM was obtained (Table 2).

### 1.2) Phosphorylation Contributes Only 30% of the Standard Binding Free Energy

Association of SHP2 domains with the partner proteins is regulated by phosphorylation, and therefore the phosphate group is necessarily responsible for a large fraction of the binding affinity. On the other hand, in order to have a good selectivity, the rest of the sequence must also contribute significantly to the peptide/protein interaction. To quantify this aspect, authors performed a binding experiment (FIG. 2) with an unphosphorylated analog of the labeled IRS-1 pY1172 peptide, CF-P9Y0 (Table 1). The affinity was approximately 100 times lower, with a KD of 6.6±0.6 µM, compared with 53±2 nM for the phosphorylated peptide. The corresponding values for the standard free energy of binding (assuming a 1 M standard state) are -29.6±0.2 kJ/mol and -41.6±0.1 kJ/mol, respectively. Assuming additivity of contributions, the phosphate group results to be responsible for the difference of -12.0±0.2 kJ/mol, i.e. for less than 30% of the total standard binding free energy of the phosphorylated peptide. This result indicates that the contribution of the rest of the peptide predominates in the binding interactions.

### 2) Sequence Optimization

### 2.1) The Sequence Can Be Reduced to 8 Amino Acids Without Loss in Affinity

Literature data are partially contradictory regarding the effect of shortening the IRS-1 pY1172 sequence on the binding affinity. Kay [1998] reported that the sequence could be shortened at the C-terminus down to the +5 residue without any loss in affinity. By contrast, Case [1994] observed a significant reduction in affinity residues in determining the N-SH2 domain binding affinity, authors performed displacement studies (FIG. 3) with unlabeled peptide P9, and with the shortened analogues P8 and P7 (Table 1), where residues -3 or -2 and -3 were removed, respectively. No significant loss in affinity was observed by reducing the sequence to 8 residues, while elimination of the amino acid at position -2 caused a drastic perturbation of complex stability (FIG. 3). The -2 to +5 IRS-1 sequence is the minimal peptide with a low nM dissociation constant.

### 2.2) Single Amino Acids Substitutions Improve the K_{d} to the Low nM Range.

Based on the structures of phosphopeptide/N-SH2 complexes [Lee 1994, Hayashi 2017] the IRS-1 pY1172 sequence is expected to have several favorable interactions, since it has apolar residues at positions +1, +3 and +5, which point towards the hydrophobic groove in the N—SH2 sequence, and anionic amino acids at positions +2 and +4, which can interact with K residues in the BG loop.

In an effort to further optimize the binding affinity, authors have analyzed in silico the effect of different apolar amino acids in position +5. Free energy calculations indicated that substitution of L with the bulkier W (but not with F) could be favorable (FIG. 4). The additional substitution of D in +4 with the longer E was evaluated as well, in view of a possible strengthening of the electrostatic interactions. In this case, no further increase in binding affinity was predicted (FIG. 4). Analogs with F or W at position +5 (P8F5 and P8W5), as well as a labeled analog with the L to W substitution (CF-P9W5, Table 1) were synthesized and studied experimentally (FIG. 5). As predicted, introduction of W in +5 was highly favorable, leading to reduction in the dissociation constant by an order of magnitude, both for the labeled and the unlabeled analog (Tables 2 and 3). As a consequence, the K_{d} for the P8W5 analog was 1.6±0.4 nM. By contrast, the additional D to E substitution resulted in a slight loss in binding affinity (FIG. 5 and Table 2 and 3).

Based on these results, further studies concentrated on the peptide with W in position +5.

### 3) Binding Selectivity

### 3.1) The Modified Sequence Is Highly Selective for the N-SH2 Domain of SHP2.

The selectivity of binding of CF-P9W5 was first assessed with respect to the C-SH2 domain of SHP2, again with the fluorescence anisotropy assay (FIG. 6). As reported in Table 2, the affinity for the C-SH2 domain was almost 1000 times less than that for the N-SH2 domain.

A more complete analysis of the binding selectivity was performed on a protein array of 97 human SH2 domains (FIG. 7). An analogue of CF-P9W5 was employed in this assay, where CF was substituted with the Cy3 dye, suitable for detection in the array reader. Control binding experiments showed that the change in fluorophore did affect peptide binding affinity only marginally (Table 2).

Strikingly, significant binding was observed only with the N-SH2 domain of SHP2, and, to a lesser extent, to the SH2 domain of the adapter protein APS (also called SHP2B2). It is worth noting that binding to the N-SH2 domain of SHP1, which has the highest identity with that of SHP2 [Liu 2006], was negligible, too.

### 4) Engineering Resistance to Degradation

### 4.1) Introduction of a Non-hydrolysable pY Mimic Is Compatible With Low nM Binding Affinity

In view of intracellular or in vivo applications of the peptide, it is essential to make it resistant to degradation. The most labile moiety is the phosphate group of the pY residue, which can be hydrolyzed by protein tyrosine phosphatase, possibly also including SHP2, of which IRS-1 pY 1172 has been shown to be a substrate [Noguchi 1994]. Authors substituted the pY with the non-hydrolysable mimetic phosphonodifluoromethyl phenylalanine (F₂Pmp), which is isosteric with pY and has a total negative charge comparable to that of pY under physiologic pH conditions [Burke 2006].

Binding experiments demonstrated that the substituted analogue (CF-P9ND0W5, where ND stands for non-dephosphorylatable, Table 1) has a dissociation constant for the N-SH2 domain which is just an order of magnitude lower with respect to that of CF-P9W5 (68 ± 5 nM with respect to 4.6 ± 0.4 nM) (FIG. 8 and Table 2). Similarly, the dissociation constant for the unlabeled peptide P9ND0W5 was 15 ± 0.4 nM (with respect to 1.6 ± 0.4 nM for P8W5) and thus remained in the nM range (Table 3).

For the sake of brevity, in the following text, CF-P9ND0W5 and its unlabeled analogue P9ND0W5 will be also referred to as the optimized peptides, or CF-OP and OP, respectively.

### 4.2) The Optimized Peptide OP Is Resistant to Proteolytic Degradation

To test the resistance to proteases, the optimized peptide OP was incubated in human serum for up to 90 minutes, or in DMEM for three days, and then analyzed by HPLC. No significant degradation was observed in these time frames (FIG. 9). By contrast, the octadecapeptide HPA3NT3 [Park 2008], which authors used as a positive control, was completely degraded already after 5 minutes (data not shown). This result is important for in vivo applications of the peptide.

### 5) Binding to and Activation of the Whole SHP2 Protein

### 5.1) OP Binds to Pathogenic Mutants With Much Higher Affinity Than to the WT

As discussed in the introduction, authors and others have hypothesized that in the autoinhibited state the conformation of the N-SH2 domain prevents efficient association to binding partners, while the binding affinity to phosphorylated sequences is maximized in the open, active state [Keilhack 2005; Bocchinfuso 2007, Martinelli 2008, LaRochelle 2018]. This model has many relevant consequences, because it implies that pathogenic mutants have a twofold effect: they increase the activity of the phosphatase, but also its affinity towards binding partners. In principle, both effects could be the origin of the hyperactivation of the signal transduction pathways involved in the pathologies caused by PTPN11 mutations.

Notwithstanding the relevance of this aspect, to the best of our knowledge, no direct phosphopeptide binding experiments to the whole SHP2 protein have ever been performed, possibly due to the fact that pY can be dephosphorylated by the PTP domain. Now, peptide OP and its fluorescent analogue CF-OP allow us to directly assess the hypothesis described above.

FIG. 10 and Table 2 report the results of binding experiments performed with CF-OP and WT SHP2 or the pathogenic mutants A72S, E76K, D61H, F71L and E76V. E76K is among the most common lesions associated with leukemia, and has never been observed in individuals with Noonan syndrome (NS). This mutation is strongly activating, with a basal activity of the corresponding mutant being at least 10 times higher than that of the WT protein. Conversely, A72S specifically recurs among subjects with NS. In this case, basal activation is only twofold [Bocchinfuso 2007]. Mutations D61H, F71L and E76V have been identified as somatic events in haematopoietic and lymphoid neoplasms [Tartaglia 2003, Tartaglia 2005]. Interestingly, authors observed that the affinity for CF-OP nicely parallels the basal activity of these mutants (FIG. 10). This finding provides a first direct confirmation that the closed, autoinhibited state has a lower affinity for the binding partners, compared to the open, active conformation.

The present data have also another important consequence: in a cellular environment, the peptide would act as an effective inhibitor of the protein-protein interactions of mutant, hyperactivated SHP2, while it would leave essentially unperturbed the WT protein. This behavior is the exact opposite of what has been observed for allosteric inhibitors, such as SHP099, which have a significantly impaired activity in pathogenic variants of SHP2 [Sun 2018; LaRochelle 2018].

### 5.2) OP Is Also an Inhibitor of the PTP Domain.

Based on previous reports of the dephosphorylation of IRS-1 pY 1172 by SHP2 [Noguchi 1994], authors verified if P8 and P8W5 are also a substrate of this protein. As reported in FIG. 11, dephosphorylation was indeed observed, although to a lower extent than for other phosphopeptides.

Using the non-dephosphorylatable peptide CD-OP, authors measured directly binding to the PTP domain of SHP2 (FIG. 12). Significant association was observed, although with a much lower affinity than with the N-SH2 domain (K_{d}=10.0 ± 0.8 µM). This finding indicates that in principle, the non-dephosphorylatable OP could act as a double hit SHP2 inhibitor, acting on both protein-protein interactions and catalytic activity.

### 5.3) OP Activates SHP2 Only Weakly

SHP2 activation is caused by binding of mono- or bi-phosphorylated peptides. Authors tested the effect of OP on the WT protein, or on the A72S mutant (this experiment is not possible with E76K, as in that case the protein is essentially fully activated also in the absence of phosphopeptides). As shown in FIG. 13, activation was very weak, compared with that induced by the biphosphorylated BTAM peptide, and a significant effect was observed only with the mutant protein. Interestingly, under the experimental conditions used, both the WT and the A72S proteins should be nearly saturated by the OP, according to the binding experiments reported in FIG. 10. A possible explanation of this result is provided by the combination of two effects: opening of the protein, by peptide association to the N-SH2 domain, and partial inhibition of the catalytic activity by association to the phosphatase domain. This finding is interesting, as SHP2 activation by the OP might have been an undesirable side effect. The Authors’ findings indicate that the peptide can inhibit SHP2 protein-protein interactions, without causing a significant increase in catalytic activity.

### 6) Cell Uptake

### 6.1) Cell Penetrating Peptides Deliver the Phosphopeptides Intracellularly, Efficiently and Without Toxicity.

SHP2 is an intracellular target, but spontaneous cell uptake of the highly charged peptides developed here is highly unlikely. To solve this issue, authors developed two different strategies, involving cell-penetrating peptide (CPP) sequences. These peptides are able to spontaneously cross the cell membranes, carrying with them macromolecular cargoes to which they have been associated covalently or non covalently. For the non covalent strategy, authors simply mixed the peptides with the Pep1 CPP [Bobone 2011]. Authors also covalently linked the sequence of fragment 48-57 of the human immunodeficiency virus type 1 TAT to the C-terminus of the peptides (Table 1) [Brooks 2005].

Fluorescence-activated cell sorting (FACS) experiments on NIH3T3 cells with the CF-P9W5 peptide demonstrated that indeed spontaneous uptake is minimal. By contrast, cell uptake could be efficiently obtained with both the covalent and the non-covalent strategy (FIG. 14). In both cases, a significant, dose dependent intracellular concentration of labeled peptide was detected. No saturation effects were observed in the concentration range investigated. Time-course experiments showed that, even after 2 hours of incubation, cell uptake was not complete (FIG. 14).

The concentration of Pep1 to be used in the delivery studies was determined based on preliminary experiments, showing that cell uptake was maximal at 10 µM CPP concentration, irrespective of the cargo concentration (FIG. 15). Under these conditions, Pep1 toxicity was negligible. CF-P9W5 TAT did not cause any cell killing at all the concentrations tested (FIG. 16).

### 7) OP Effectively Reverses the Effects of D61G Mutation in Vivo

The zebrafish model system was used to explore the in vivo effect of the peptide. Zebrafish SHP2a is highly homologous to human SHP2 (91.2% protein sequence identity); in particular, the sequence of the N-SH2 domain and of the N-SH2/PTP interface are identical in the human and fish proteins. RASopathies-associated mutants, including activating mutants of Shp2a, greatly impact zebrafish development. In humans, the D61G substitution has been found in both NS and leukemia [Kratz 2005] and in animal models it induces both NS-like features and myeloproliferative disease [Araki 2004]. Microinjection of synthetic mRNA encoding NS-associated mutants of Shp2 at the one-cell stage induces NS-like traits [Jopling 2007]. During gastrulation, convergence and extension movement are affected, resulting in oval-shaped embryos, with increased major/minor axis length ratio at 11 hpf [Jopling 2007]. Inventors coinjected Shp2a-D61G mRNA with OP in zebrafish embryos, to investigate whether OP rescues the defective cell movements during gastrulation [Bobone 2020]. As shown in FIG. 7, a dose-dependent decrease in Shp2a-D61G induced major/minor axis ratios was observed, with a rescue of the phenotype that was significant at 5 µM peptide concentration. On the other hand, embryos injected with Shp2a-WT were almost perfect spheres at 11 hpf, and co-injection with 3 µM peptide had no impact on their shape. As expected, a large portion of Shp2a-D61G injected embryos were severely affected and they died during embryonic development, whereas injection of WT Shp2a did not induce significant lethality. Following the survival of Shp2a-D61G injected embryos, a significant and dose dependent improvement in the survival of embryos was observed upon co-injection with 0.3 µM, 3 µM and 5 µM OP. By contrast, lethality of WT Shp2a embryos was not affected by co-injection of 3 µM OP. Altogether, these results indicate that co-injection of the OP rescued the developmental defects induced by a pathogenic, basally activated Shp2a variant, while it had no effect on WT embryos.

### Tables

TABLE 1

Peptide sequences investigated in the invention

| Abbreviation | Sequence | Rₜ (min) | [M+H]⁺ | Purity | SEQ ID NO: |
|---|---|---|---|---|---|
| P9 | GLN-pY-IDLDL | 21.2 (10-40%B in 30′) | 1156.5 | 95% | 2 |
| P9Y0 | GLN- Y-IDLDL | 10.9 (20-50%B in 30′) | 1076.6 | 99% | 12 |
| P8 | LN-pY-IDLDL | 24.3 (10-40 %B in 30′) | 1099.4 | 92% | 13 |
| P7 | N-pY-IDLDL | 16.9 (10-40 %B in 30′) | 986.3 | 92% | 14 |
| P8W5 | LN-pY-IDLDW | 12.5 (20-60%B in 20′) | 1172.5 | 95% | 15 |
| P8F5 | LN-pY-IDLDF | 16.5 (10-95%B in 30′) | 1133.5 | 93% | 16 |
| P8E4W5 | LN-pY-IDLEW | 12.4 (20-60%B in 20′) | 1186.5 | 98% | 17 |
| P9ND0W5 (or OP) | GLN-F₂Pmp-IDLDW | 15.7 (10-50%B in 20′) | 1263.4 | 94% | 5 |
| CF-P9 | CF-GLN-pY-IDLDL | 14.9 (20-50%B in 30′) | 1473.5 | 93% | 18 |
| CF-P9Y0 | CF-GLN- Y-IDLDL | 18.7 (20-50%B in 30′) | 1392.6 | 99% | 19 |
| CF-P9W5 | CF-GLN-pY-IDLDW | 14.0 (20-60%B in 20′) | 1545.5 | 96% | 20 |
| Cy3-P9W5 | Cy3-GLN-pY-IDLDW | 19.6 (20-60%B in 20′) | 1626.7 | 95% | 21 |
| CF-P9E4W5 | CF-GLN-pY-IDLEW | 13.9 (20-60%B in 20′) | 1559.6 | 97% | 22 |
| CF-P9ND0W5 (or CF-OP) | CF-GLN-F₂Pmp-IDLDW | 16.1 (5-65%B in 20′) | 1580.4 | 93% | 23 |
| P8W5-TAT | LN-pY-IDLDW-GRKKRRQRRR | 16.1 (5-65%B in 30′) | 2550.4 | 95% | 24 |
| CF-P9W5-TAT | CF-GLN-pY-IDLDW-GRKKRRQRRR | 18.2 (5-65%B in 30′) | 2924.6 | 90% | 10 |

All peptides were amidated at the C-terminus. Unlabeled peptides were acetylated at the N-terminus. CF is 5,6 carboxyfluorescein, Cy3 is Cyanine 3 carboxylic acid and F₂Pmp is the non-dephosphorylatable pY mimic phosphonodifluoromethyl phenylalanine . Retention times (Rt) for the synthetic peptides obtained from RP-HPLC (the different elution conditions used for the various peptides are shown in brackets) and molecular weights for the synthetic peptides experimentally determined by ESI-MS spectrometry, and purities are reported.

TABLE 2

Dissociation constants obtained from the fluorescence anisotropy binding experiments.

| Peptide | Domain/Protein | K_{D} (nM) |
|---|---|---|
| CF-P9 | N-SH2 | 53 ± 2 |
| CF-P9Y0 | N-SH2 | 6600 ± 600 |
| CF-P9W5 | N-SH2 | 4.6 ± 0.4 |
| CF-P9W5 | C-SH2 | 4200 ± 300 |
| Cy3-P9W5 | N-SH2 | 23 ± 2 |
| CF-P9E4W5 | N-SH2 | 8.2 ± 0.7 |
| CF-P9ND0W5 (CF-OP) | N-SH2 | 68 ± 5 |
| CF-P9ND0W5 (CF-OP) | PTP | 10000 ± 800 |
| CF-P9ND0W5 (CF-OP) | WT | 930 ± 70 |
| CF-P9ND0W5 (CF-OP) | A72S | 400 ± 40 |
| CF-P9ND0W5 (CF-OP) | E76V | 330 ± 10 |
| CF-P9ND0W5 (CF-OP) | D61H | 170 ± 10 |
| CF-P9ND0W5 (CF-OP) | F71L | 140 ± 10 |
| CF-P9ND0W5 (CF-OP) | E76K | 48 ± 2 |

TABLE 3

Inhibition constants obtained from the displacement experiments. All the measurements were performed on the N-SH2 domain of SHP2. Experiments were performed at [N-SH2]= 3.4 nM and [CF-P9W5]=0.5 nM (for P8 and P9ND0W5) or 0.1 nM (for the other peptides)

| Peptide | IC₅₀ (nM) | Kᵢ (nM) |
|---|---|---|
| P8 | 47 ± 4 | 25 ± 4 |
| P8F5 | 16 ± 1 | 9 ± 2 |
| P8W5 | 5.4 ± 0.3 | 1.6 ± 0.4 |
| P8E4W5 | 11 ± 1 | 5 ± 2 |
| P9ND0W5 | 32 ± 5 | 15 ± 5 |

### References

Abraham, M.J. et al. (2015) GROMACS: High performance molecular simulations through multi-level parallelism from laptops to supercomputers. SoftwareX 1-2, 19-25.

Ahmed, T. A., Adamopoulos, C., Karoulia, Z., Wu, X., Sachidanandam, R., Aaronson, S. A., & Poulikakos, P. I. (2019). SHP2 Drives Adaptive Resistance to ERK Signaling Inhibition in Molecularly Defined Subsets of ERK-Dependent Tumors. Cell reports, 26(1), 65-78.

Araki, T., Mohi, M. G., Ismat, F. A., Bronson, R. T., Williams, I. R., Kutok, J. L., ... & Neel, B. G. (2004). Mouse model of Noonan syndrome reveals cell type-and gene dosage-dependent effects of Ptpn11 mutation. Nature medicine, 10(8), 849-857.

Barlow, R., & Blake, J. F. (1989). Hill coefficients and the logistic equation. Trends in pharmacological sciences, 10(11), 440-441.

Bentires-Alj M, Paez JG, David FS, Keilhack H, Halmos B, Naoki K, Maris JM, Richardson A, Bardelli A, Sugarbaker DJ et al.: Activating mutations of the Noonan Syndrome-associated SHP2/PTPN11 gene in human solid tumors and adult acute myelogenous leukemia. Cancer Res 2004, 64:8816-8820.

Blaskovich, M. A. (2009). Drug discovery and protein tyrosine phosphatases. Current medicinal chemistry, 16(17), 2095-2176.

Bobone, S., Piazzon, A., Orioni, B., Pedersen, J. Z., Nan, Y. H., Hahm, K. S., ... & Stella, L. (2011). The thin line between cell-penetrating and antimicrobial peptides: the case of Pep-1 and Pep-1-K. Journal of Peptide Science, 17(5), 335-341.

Bocchinfuso, G., Stella, L., Martinelli, S., Flex, E., Carta, C., Pantaleoni, F., ... & Palleschi, A. (2007). Structural and functional effects of disease-causing amino acid substitutions affecting residues Ala72 and Glu76 of the protein tyrosine phosphatase SHP-2. Proteins: Structure, Function, and Bioinformatics, 66(4), 963-974.

Bolton, A. E., & Hunter, W. M. (1973). The labelling of proteins to high specific radioactivities by conjugation to a 125I-containing acylating agent. Application to the radioimmunoassay. Biochemical Journal, 133(3), 529-538.

Bradford, M. M. (1976). A rapid and sensitive method for the quantitation of microgram quantities of protein utilizing the principle of protein-dye binding. Analytical biochemistry, 72(1-2), 248-254.

Brooks, H., Lebleu, B., & Vives, E. (2005). Tat peptide-mediated cellular delivery: back to basics. Advanced drug delivery reviews, 57(4), 559-577.

Bunda, S., Burrell, K., Heir, P., Zeng, L., Alamsahebpour, A., Kano, Y., ... & Ohh, M. (2015). Inhibition of SHP2-mediated dephosphorylation of Ras suppresses oncogenesis. Nature communications, 6, 8859.

Burke, J. T. (2006). Design and synthesis of phosphonodifluoromethyl phenylalanine (F2Pmp): a useful phosphotyrosyl mimetic. Current topics in medicinal chemistry, 6(14), 1465-1471. Bussi, G., Donadio, D., & Parrinello, M. (2007). Canonical sampling through velocity rescaling. The Journal of chemical physics, 126(1), 014101.

Butterworth, S., Overduin, M., & Barr, A. J. (2014). Targeting protein tyrosine phosphatase SHP2 for therapeutic intervention. Future medicinal chemistry, 6(12), 1423-1437.

Case, R. D., Piccione, E., Wolf, G., Benett, A. M., Lechleider, R. J., Neel, B. G., & Shoelson, S. E. (1994). SH-PTP2/Syp SH2 domain binding specificity is defined by direct interactions with platelet-derived growth factor beta-receptor, epidermal growth factor receptor, and insulin receptor substrate-1-derived phosphopeptides. Journal of Biological Chemistry, 269(14), 10467-10474.

Castanho, M. A., Santos, N. C., & Loura, L. M. (1997). Separating the turbidity spectra of vesicles from the absorption spectra of membrane probes and other chromophores. European biophysics journal, 26(3), 253-259.

Chen, Y. N. P., LaMarche, M. J., Chan, H. M., Fekkes, P., Garcia-Fortanet, J., Acker, M. G., ... & Dobson, J. R. (2016). Allosteric inhibition of SHP2 phosphatase inhibits cancers driven by receptor tyrosine kinases. Nature, 535(7610), 148.

Chovancova, E., Pavelka, A., Benes, P., Stmad, O., Brezovsky, J., Kozlikova, B., ... & Biedermannova, L. (2012). CAVER 3.0: a tool for the analysis of transport pathways in dynamic protein structures. PLoS computational biology, 8(10), e1002708.

Dardaei, L., Wang, H. Q., Singh, M., Fordjour, P., Shaw, K. X., Yoda, S., ... & Chen, Y. (2018). SHP2 inhibition restores sensitivity in ALK-rearranged non-small-cell lung cancer resistant to ALK inhibitors. Nature medicine, 24(4), 512.

Darden, T., York, D., & Pedersen, L. (1993) Particle mesh Ewald: An N· log (N) method for Ewald sums in large systems. The Journal of chemical physics, 98(12), 10089-10092.

Digilio, M. C., Conti, E., Sarkozy, A., Mingarelli, R., Dottorini, T., Marino, B., ... & Dallapiccola, B. (2002). Grouping of multiple-lentigines/LEOPARD and Noonan syndromes on the PTPN11 gene. The American Journal of Human Genetics, 71(2), 389-394.

Dunbrack Jr, R. L. (2002). Rotamer libraries in the 21st century. Current opinion in structural biology, 12(4), 431-440.

Elson, A. (2018). Stepping out of the shadows: Oncogenic and tumor-promoting protein tyrosine phosphatases. The international journal of biochemistry & cell biology, 96, 135-147.

Esbjörner, E. K., Lincoln, P., & Norden, B. (2007). Counterion-mediated membrane penetration: cationic cell-penetrating peptides overcome Born energy barrier by ion-pairing with phospholipids. Biochimica et Biophysica Acta (BBA)-Biomembranes, 1768(6), 1550-1558.

Espejo, A., Jocelyn, C. Ô. T. E., Bednarek, A., Richard, S., & Bedford, M. T. (2002). A protein-domain microarray identifies novel protein-protein interactions. Biochemical Journal, 367(3), 697-702.

Fabrini, R., De Luca, A., Stella, L., Mei, G., Orioni, B., Ciccone, S., ... & Ricci, G. (2009). Monomer- dimer equilibrium in glutathione transferases: a critical re-examination. Biochemistry, 48(43), 10473-10482.

Fedele, C., Ran, H., Diskin, B., Wei, W., Jen, J., Geer, M. J., ... & Neel, B. G. (2018). SHP2 inhibition prevents adaptive resistance to MEK inhibitors in multiple cancer models. Cancer discovery, 8(10), 1237-1249.

Frankson, R., Yu, Z. H., Bai, Y., Li, Q., Zhang, R. Y., & Zhang, Z. Y. (2017). Therapeutic targeting of oncogenic tyrosine phosphatases. Cancer research, 77(21), 5701-5705.

Garcia Fortanet, J., Chen, C. H. T., Chen, Y. N. P., Chen, Z., Deng, Z., Firestone, B., ... & Grunenfelder, D. (2016). Allosteric inhibition of SHP2: identification of a potent, selective, and orally efficacious phosphatase inhibitor. Journal of medicinal chemistry, 59(17), 7773-7782.

Gelb, B. D., Roberts, A. E., & Tartaglia, M. (2015). Cardiomyopathies in Noonan syndrome and the other RASopathies. Progress in pediatric cardiology, 39(1), 13-19.

Grosskopf, S., Eckert, C., Arkona, C., Radetzki, S., Böhm, K., Heinemann, U., ... & Rademann, J. (2015). Selective inhibitors of the protein tyrosine phosphatase SHP2 block cellular motility and growth of cancer cells in vitro and in vivo. ChemMedChem, 10(5), 815-826.

Grossmann, K. S., Rosario, M., Birchmeier, C., & Birchmeier, W. (2010). The tyrosine phosphatase Shp2 in development and cancer. In Advances in cancer research (Vol. 106, pp. 53-89). Academic Press.

Gu, S., Sayad, A., Chan, G., Yang, W., Lu, Z., Virtanen, C., ... & Neel, B. G. (2018). SHP2 is required for BCR-ABL1-induced hematologic neoplasia. Leukemia, 32(1), 203.

Hayashi, T., Senda, M., Suzuki, N., Nishikawa, H., Ben, C., Tang, C., ... & Hatakeyama, M. (2017). Differential mechanisms for SHP2 binding and activation are exploited by geographically distinct Helicobacter pylori CagA oncoproteins. Cell reports, 20(12), 2876-2890.

Hess, B; Bekker H; Berendsen HJC; Fraaije JGEM. (1997) LINCS: A Linear Constraint Solver for Molecular Simulations. Journal of Computational Chemistry. 18 (12): 1463-1472.

Higashi, H., Tsutsumi, R., Muto, S., Sugiyama, T., Azuma, T., Asaka, M., & Hatakeyama, M. (2002). SHP-2 tyrosine phosphatase as an intracellular target of Helicobacter pylori CagA protein. Science, 295(5555), 683-686.

Hill, K. S., Roberts, E. R., Wang, X., Marin, E., Park, T. D., Son, S., ... & Wan, L. (2019). PTPN11 plays oncogenic roles and is a therapeutic target for BRAF wild-type melanomas. Molecular Cancer Research, 17(2), 583-593.

Hof, P., Pluskey, S., Dhe-Paganon, S., Eck, M. J., & Shoelson, S. E. (1998). Crystal structure of the tyrosine phosphatase SHP-2. Cell, 92(4), 441-450.

Homeyer, N., Horn, A. H., Lanig, H., & Sticht, H. (2006). AMBER force-field parameters for phosphorylated amino acids in different protonation states: phosphoserine, phosphothreonine, phosphotyrosine, and phosphohistidine. Journal of molecular modeling, 12(3), 281-289.

Huang, H., Li, L., Wu, C., Schibli, D., Colwill, K., Ma, S., ... & Pawson, T. (2008). Defining the specificity space of the human SRC homology 2 domain. Molecular & Cellular Proteomics, 7(4), 768-784.

Huang, X. (2003). Fluorescence polarization competition assay: the range of resolvable inhibitor potency is limited by the affinity of the fluorescent ligand. Journal of biomolecular screening, 8(1), 34-38.

Hub, J. S., De Groot, B. L., & Van Der Spoel, D. (2010). g_wham - A Free Weighted Histogram Analysis implementation Including Robust Error and Autocorrelation Estimates. Journal of chemical theory and computation, 6(12), 3713-3720.

Jopling, C., van Geemen, D., & den Hertog, J. (2007). Shp2 knockdown and Noonan/LEOPARD mutant Shp2-induced gastrulation defects. PLoS genetics, 3(12), e225.

Jorgensen, W. L., Chandrasekhar, J., Madura, J. D., Impey, R. W., & Klein, M. L. (1983). Comparison of simple potential functions for simulating liquid water. The Journal of chemical physics, 79(2), 926-935.

Kay, L. E, Muhandiram, D. R., Wolf, G., Shoelson, S. E., & Forman-Kay, J. D. (1998). Correlation between binding and dynamics at SH2 domain interfaces. Nature structural biology, 5(2), 156.

Keilhack, H., David, F. S., McGregor, M., Cantley, L. C., & Neel, B. G. (2005). Diverse Biochemical Properties of Shp2 Mutants. Implications for Disease Phenotypes. Journal of Biological Chemistry, 280(35), 30984-30993.

Kertesz, A., Varadi, G., Toth, G. K., Fajka-Boja, R., Monostori, E., & Sarmay, G. (2006 a). Optimization of the cellular import of functionally active SH2-domain-interacting phosphopeptides. Cellular and Molecular Life Sciences CMLS, 63(22), 2682-2693.

Kertesz, A., Takacs, B., Varadi, G., Toth, G. K., & Sarmay, G. (2006 b). Design and Functional Activity of Phosphopeptides with Potential Immunomodulating Capacity, Based on the Sequence of Grb2-Associated Binder 1. Annals of the New York Academy of Sciences, 1091(1), 437-444.

Kratz, C. P., Niemeyer, C. M., Castleberry, R. P., Cetin, M., Bergsträsser, E., Emanuel, P. D., ... & Stary, J. (2005). The mutational spectrum of PTPN11 in juvenile myelomonocytic leukemia and Noonan syndrome/myeloproliferative disease. Blood, 106(6), 2183-2185.

Kumar S., Rosenberg J.M., Bouzida D., Swendsen R.H., Kollman P.A. (1992) The weighted histogram analysis method for free-energy calculations on biomolecules. I. The method. J. Comput. Chem. 13:1011-1021.

Ladbury, J. E., Lemmon, M. A., Zhou, M., Green, J., Botfield, M. C., & Schlessinger, J. (1995). Measurement of the binding of tyrosyl phosphopeptides to SH2 domains: a reappraisal. Proceedings of the National Academy of Sciences, 92(8), 3199-3203.

LaRochelle, J. R., Fodor, M., Vemulapalli, V., Mohseni, M., Wang, P., Stams, T., ... & Blacklow, S. C. (2018). Structural reorganization of SHP2 by oncogenic mutations and implications for oncoprotein resistance to allosteric inhibition. Nature communications, 9(1), 4508.

Lee, C. H., Kominos, D., Jacques, S., Margolis, B., Schlessinger, J., Shoelson, S. E., & Kuriyan, J. (1994). Crystal structures of peptide complexes of the amino-terminal SH2 domain of the Syp tyrosine phosphatase. Structure, 2(5), 423-438.

Legius, E., Schrander-Stumpel, C., Schollen, E., Pulles-Heintzberger, C., Gewillig, M., & Fryns, J. P. (2002). PTPN11 mutations in LEOPARD syndrome. Journal of medical genetics, 39(8), 571-574.

Liu, B. A., Jablonowski, K., Raina, M., Arce, M., Pawson, T., & Nash, P. D. (2006). The human and mouse complement of SH2 domain proteins—establishing the boundaries of phosphotyrosine signaling. Molecular cell, 22(6), 851-868.

Loh ML, Martinelli S, Cordeddu V, Reynolds MG, Vattikuti S, Lee CM, Wulfert M, Germing U, Haas P, Niemeyer C, Beran ME, Strom S, Lübbert M, Sorcini M, Estey EH, Gattermann N, Tartaglia M. Acquired PTPN11 mutations occur rarely in adult patients with myelodysplastic syndromes and chronic myelomonocytic leukemia. Leuk Res. 2005 Apr;29(4):459-62.

Lu, H., Liu, C., Velazquez, R., Wang, H., Dunkl, L. M., Kazic-Legueux, M., ... & Moody, S. (2019). SHP2 inhibition overcomes RTK-mediated pathway re-activation in KRAS mutant tumors treated with MEK inhibitors. Molecular cancer therapeutics, molcanther-0852.

Machida, K., & Mayer, B. J. (2005). The SH2 domain: versatile signaling module and pharmaceutical target. Biochimica et Biophysica Acta (BBA)-Proteins and Proteomics, 1747(1), 1-25.

Mainardi, S., Mulero-Sanchez, A., Prahallad, A., Germano, G., Bosma, A., Krimpenfort, P., ... & Nadal, E. (2018). SHP2 is required for growth of KRAS-mutant non-small-cell lung cancer in vivo. Nature medicine, 24(7), 961.

Martinelli, S., Torreri, P., Tinti, M., Stella, L., Bocchinfuso, G., Flex, E., ... & Castagnoli, L. (2008). Diverse driving forces underlie the invariant occurrence of the T42A, E139D, I282V and T468M SHP2 amino acid substitutions causing Noonan and LEOPARD syndromes. Human molecular genetics, 17(13), 2018-2029.

Martinelli S, Carta C, Flex E, Binni F, Cordisco EL, Moretti S, Puxeddu E, Tonacchera M, Pinchera A, McDowell HP, Dominici C, Rosolen A, Di Rocco C, Riccardi R, Celli P, Picardo M, Genuardi M, Grammatico P, Sorcini M, Tartaglia M. Activating PTPN11 mutations play a minor role in pediatric and adult solid tumors. Cancer Genet Cytogenet. 2006 Apr 15;166(2):124-9.

Martinelli S., McDowell HP, Vigne SD, Kokai G, Uccini S, Tartaglia M, Dominici C. RAS signaling dysregulation in human embryonal Rhabdomyosarcoma. Genes Chromosomes Cancer. 2009 Nov;48(11):975-82.

Martinelli, S., Nardozza, A. P., Delle Vigne, S., Sabetta, G., Torreri, P., Bocchinfuso, G., ... & Cesareni, G. (2012). Counteracting effects operating on Src homology 2 domain-containing protein-tyrosine phosphatase 2 (SHP2) function drive selection of the recurrent Y62D and Y63C substitutions in Noonan syndrome. Journal of Biological Chemistry, 287(32), 27066-27077.

Martinelli S, Pannone L, Lissewski C, J. Brinkmann, E. Flex, D. Schanze, P. Calligari, M. Anselmi, F. Pantaleoni, V. C. Canale, A. Ioannides, N. Rahner, D. Josifova, G. Bocchinfuso, M. Ryten, L. Stella, M. Tartaglia, M. Zenker. Pathogenic PTPN11 variants involving the poly-glutamine Gln255 -Gln256 -Gln257 stretch highlight the relevance of helix B in SHP2’s functional regulation. Hum. Mut., 2020, DOI: 10.1002/humu.24007.

Nichols, R. J., Haderk, F., Stahlhut, C., Schulze, C. J., Hemmati, G., Wildes, D., ... & Hsieh, T. (2018). RAS nucleotide cycling underlies the SHP2 phosphatase dependence of mutant BRAF-, NF1-and RAS-driven cancers. Nature cell biology, 20(9), 1064.

Niemeyer, C. M., & Flotho, C. (2019). Juvenile myelomonocytic leukemia: who’s the driver at the wheel?. Blood, 133(10), 1060-1070.

Nikolovska-Coleska, Z., Wang, R., Fang, X., Pan, H., Tomita, Y., Li, P., ... & Wang, S. (2004). Development and optimization of a binding assay for the XIAP BIR3 domain using fluorescence polarization. Analytical biochemistry, 332(2), 261-273.

Noguchi, T., Matozaki, T., Horita, K., Fujioka, Y., & Kasuga, M. (1994). Role of SH-PTP2, a protein-tyrosine phosphatase with Src homology 2 domains, in insulin-stimulated Ras activation. Molecular and cellular biology, 14(10), 6674-6682.

Okazaki, T., Chikuma, S., Iwai, Y., Fagarasan, S., & Honjo, T. (2013). A rheostat for immune responses: the unique properties of PD-1 and their advantages for clinical application. Nature immunology, 14(12), 1212.

Pace, C. N., Vajdos, F., Fee, L., Grimsley, G., & Gray, T. (1995). How to measure and predict the molar absorption coefficient of a protein. Protein science, 4(11), 2411-2423.

Pannone, L., Bocchinfuso, G., Flex, E., Rossi, C., Baldassarre, G., Lissewski, C., ... & Anselmi, M. (2017). Structural, functional, and clinical characterization of a novel PTPN11 mutation cluster underlying Noonan syndrome. Human mutation, 38(4), 451-459.

Pardoll, D. M. (2012). The blockade of immune checkpoints in cancer immunotherapy. Nature Reviews Cancer, 12(4), 252.

Park, S. C., Kim, M. H., Hossain, M. A., Shin, S. Y., Kim, Y., Stella, L., ... & Hahm, K. S. (2008). Amphipathic α-helical peptide, HP (2-20), and its analogues derived from Helicobacter pylori: pore formation mechanism in various lipid compositions. Biochimica et Biophysica Acta (BBA)-Biomembranes, 1778(1), 229-241.

Parrinello, M., & Rahman, A. (1981). Polymorphic transitions in single crystals: A new molecular dynamics method. Journal of Applied physics, 52(12), 7182-7190.

Prahallad, A., Heynen, G. J., Germano, G., Willems, S. M., Evers, B., Vecchione, L., ... & Bardelli, A. (2015). PTPN11 is a central node in intrinsic and acquired resistance to targeted cancer drugs. Cell reports, 12(12), 1978-1985.

Quintana, E., Mordec, K., Nichols, R. J., Wildes, D., Schulze, C. J., Myers, D. R.,... & Goldsmith, M. A. (2019). Abstract A103: Allosteric inhibition of SHP2 induces antitumor immunity in PD-1-sensitive tumors through modulation of both innate and adaptive mechanisms. Cancer Immunol Res 7(2 Suppl):Abstract nr A103.

Ran, H., Tsutsumi, R., Araki, T., & Neel, B. G. (2016). Sticking it to cancer with molecular glue for SHP2. Cancer Cell, 30(2), 194-196.

Roberts, A. E., Allanson, J. E., Tartaglia, M., & Gelb, B. D. (2013). Noonan syndrome. The Lancet, 381(9863), 333-342.

Roth, L., Wakim, J., Wasserman, E., Shalev, M., Arman, E., Stein, M., ... & Elson, A. (2019). Phosphorylation of the phosphatase PTPROt at Tyr399 is a molecular switch that controls osteoclast activity and bone mass in vivo. Sci. Signal., 12(563), eaau0240.

Ruess, D. A., Heynen, G. J., Ciecielski, K. J., Ai, J., Berninger, A., Kabacaoglu, D., ... & Karpathaki, A. F. (2018). Mutant KRAS-driven cancers depend on PTPN11/SHP2 phosphatase. Nature medicine, 24(7), 954.

Saxton, T. M., Henkemeyer, M., Gasca, S., Shen, R., Rossi, D. J., Shalaby, F., ... & Pawson, T. (1997). Abnormal mesoderm patterning in mouse embryos mutant for the SH2 tyrosine phosphatase Shp-2. The EMBO journal, 16(9), 2352-2364.

Schneider, C. A., Rasband, W. S., & Eliceiri, K. W. (2012). NIH Image to ImageJ: 25 years of image analysis. Nature methods, 9(7), 671-675.

Sha, F., Gencer, E. B., Georgeon, S., Koide, A., Yasui, N., Koide, S., & Hantschel, O. (2013). Dissection of the BCR-ABL signaling network using highly specific monobody inhibitors to the SHP2 SH2 domains. Proceedings of the National Academy of Sciences, 110(37), 14924-14929. Shi, Z. Q., Lu, W., & Feng, G. S. (1998). The Shp-2 tyrosine phosphatase has opposite effects in mediating the activation of extracellular signal-regulated and c-Jun NH2-terminal mitogen-activated protein kinases. Journal of Biological Chemistry, 273(9), 4904-4908.

Sun, X., Ren, Y., Gunawan, S., Teng, P., Chen, Z., Lawrence, H. R., ... & Wu, J. (2018). Selective inhibition of leukemia-associated SHP2 E69K mutant by the allosteric SHP2 inhibitor SHP099. Leukemia, 32(5), 1246.

Tajan, M., de Rocca Serra, A., Valet, P., Edouard, T., & Yart, A. (2015). SHP2 sails from physiology to pathology. European journal of medical genetics, 58(10), 509-525.

Tajan, M., Paccoud, R., Branka, S., Edouard, T., & Yart, A. (2018). The RASopathy family: consequences of germline activation of the RAS/MAPK pathway. Endocrine reviews, 39(5), 676-700.

Tartaglia, M., Mehler, E. L., Goldberg, R., Zampino, G., Brunner, H. G., Kremer, H., ... & Kalidas, K. (2001). Mutations in PTPN11, encoding the protein tyrosine phosphatase SHP-2, cause Noonan syndrome. Nature genetics, 29(4), 465.

Tartaglia, M., Niemeyer, C. M., Fragale, A., Song, X., Buechner, J., Jung, A., ... & Gelb, B. D. (2003). Somatic mutations in PTPN11 in juvenile myelomonocytic leukemia, myelodysplastic syndromes and acute myeloid leukemia. Nature genetics, 34(2), 148.

Tartaglia, M., Martinelli, S., Cazzaniga, G., Cordeddu, V., Iavarone, I., Spinelli, M., ... & Masera, G. (2004). Genetic evidence for lineage-related and differentiation stage-related contribution of somatic PTPN11 mutations to leukemogenesis in childhood acute leukemia. Blood, 104(2), 307-313.

Tartaglia, M., Martinelli, S., Iavarone, I., Cazzaniga, G., Spinelli, M., Giarin, E., ... & Locatelli, F. (2005). Somatic PTPN11 mutations in childhood acute myeloid leukaemia. British journal of haematology, 129(3), 333-339.

Tartaglia, M., Martinelli, S., Stella, L., Bocchinfuso, G., Flex, E., Cordeddu, V., ... & Sorcini, M. (2006). Diversity and functional consequences of germline and somatic PTPN11 mutations in human disease. The American Journal of Human Genetics, 78(2), 279-290.

Tartaglia, M., & Gelb, B. D. (2010). Disorders of dysregulated signal traffic through the RAS-MAPK pathway: phenotypic spectrum and molecular mechanisms. Annals of the New York Academy of Sciences, 1214, 99.

Tsutsumi, R., Ran, H., & Neel, B. G. (2018). Off-target inhibition by active site-targeting SHP 2 inhibitors. FEBS open bio, 8(9), 1405-1411.

Van de Weert, M., & Stella, L. (2011). Fluorescence quenching and ligand binding: a critical discussion of a popular methodology. Journal of Molecular Structure, 998(1-3), 144-150. Veillette, A., & Chen, J. (2018). SIRPα-CD47 immune checkpoint blockade in anticancer therapy. Trends in immunology, 39(3), 173-184.

Vuong, Q. V., Nguyen, T. T., & Li, M. S. (2015). A new method for navigating optimal direction for pulling ligand from binding pocket: application to ranking binding affinity by steered molecular dynamics. Journal of chemical information and modeling, 55(12), 2731-2738.

Wiseman, T., Williston, S., Brandts, J. F., & Lin, L. N. (1989). Rapid measurement of binding constants and heats of binding using a new titration calorimeter. Analytical biochemistry, 179(1), 131-137.

Wong, G. S., Zhou, J., Liu, J. B., Wu, Z., Xu, X., Li, T., ... & Zou, C. (2018). Targeting wild-type KRAS-amplified gastroesophageal cancer through combined MEK and SHP2 inhibition. Nature medicine, 24(7), 968.

Wu, P., Nielsen, T. E., & Clausen, M. H. (2015). FDA-approved small-molecule kinase inhibitors. Trends in pharmacological sciences, 36(7), 422-439.

Wu, X., Xu, G., Li, X., Xu, W., Li, Q., Liu, W., ... & Qu, C. K. (2019). Small Molecule Inhibitor that Stabilizes the Autoinhibited Conformation of the Oncogenic Tyrosine Phosphatase SHP2. Journal of medicinal chemistry, 62(3), 1125-1137.

Xie, J., Si, X., Gu, S., Wang, M., Shen, J., Li, H., ... & Zhu, J. (2017). Allosteric inhibitors of SHP2 with therapeutic potential for cancer treatment. Journal of medicinal chemistry, 60(24), 10205-10219.

Zhao, M., Guo, W., Wu, Y., Yang, C., Zhong, L., Deng, G., ... & Kong, L. (2019). SHP2 inhibition triggers anti-tumor immunity and synergizes with PD-1 blockade. Acta Pharmaceutica Sinica B, 9(2), 304-315.

1. A peptide comprising the sequence from N-terminus to C-terminus: X₋₂X₋₁ZX₁X₂X₃X₄X₅ (SEQ ID NO: 1) wherein
Z is a non-natural analogue of phosphotyrosine;
X-₂ is a hydrophobic amino acid;
X₋₁ is any amino acid;
X₁ is a hydrophobic amino acid;
X₃ is a hydrophobic amino acid;
X₅ is a hydrophobic amino acid; and
X₂ and X₄ are anionic amino acids;
with the proviso that the hydrophobic amino in X₅ acid is not Leu.

2. A peptide according to claim 1 comprising the sequence from N-terminus to C-terminus:
X₋₂X₋₁ZX₁X₂X₃X₄X₅ (SEQ ID NO:1)
wherein
Z is a non-natural analogue of phosphotyrosine;
X-₂ is a hydrophobic amino acid;
X₋₁ is any amino acid;
X₁ is a hydrophobic amino acid;
X₃ is a hydrophobic amino acid;
X₅ is a hydrophobic amino acid selected from the group consisting of: Trp, Phe and Tyr; and
X₂ and X₄ are anionic amino acids wherein the amino acids may be each independently a natural or non-natural amino acid .

3. A peptide according to claim 1 comprising the sequence from N- terminus to C-terminus:
X₋₂X₋₁ZX₁X₂X₃X₄X₅ (SEQ ID NO: 1)
wherein
Z is a non-natural analogue of phosphotyrosine
X-₂ is a hydrophobic amino acid,
X₋₁ is any amino acid;
X₁ is a hydrophobic amino acid;
X₃ is a hydrophobic amino acid;
X₅ is Trp; and
X₂ and X₄ are anionic amino acids wherein the amino acids may be each independently a natural or non-natural amino acid .

4. The peptide according to claim 1 wherein:
X-₂ is Leu and/or
-X₋₁ is Asn and/or
X₁ is Ile and/or
X₂ is Asp and/or
X₃ is Leu and/or
X₄ is Asp and/or
X₅ is Trp or Phe .

5. The peptide according to claim 1 wherein Z is a non-natural analogue of phosphotyrosine, .

6. The peptide according to claim 5 comprising the sequence from N-terminus to C-terminus:
LN-(F₂PmP)-IDLDW (SEQ ID NO:4) or GLN-(F₂PmP)-IDLDW (SEQ ID NO:5).

7. The peptide according to claim 1 wherein the N-terminus and/or the C-terminus of the peptide are modified.

8. The peptide according to claim 6 wherein the peptide N-terminus is acetylated, and its C-terminus is amidated.

9. A peptide according to claim 1 further comprising an aminoacidic sequence which favors penetration inside cells.

10. A non-covalent complex comprising the peptide according to claim 1 and an aminoacidic sequence which favors penetration inside cells.

11. A pharmaceutical composition comprising a peptide according to claim 1, at least one pharmaceutically acceptable carrier, excipient and/or diluent, and optionally further comprising at least one therapeutic agent.

12. The peptide according to claim 1 for use as a medicament, preferably for use:
in the treatment of childhood myeloproliferative disorders, preferably juvenile myelomonocytic leukemia (JMML), childhood myelodysplastic syndromes (e.g, RAEB), childhood leukemia (e.g, acute monocytic leukemia (AMoL, FAB M5) and acute lymphoblastic leukemia (ALL), “common” subtype), adult myelodysplastic syndromes, myelogenous and lymphoblastic leukemia, pediatric/adult solid tumors associated with an aberrant activity of SHP2 due to the occurrence of somatic PTPN11 gain-of-function mutations, e.g. neuroblastoma, glioma, embryonal rhabdomyosarcoma, lung cancer, colon cancer, and melanoma),
in the treatment of tumors associated with hyperactivation of the signal transduction pathways regulated by SHP2 (RAS-MAPK and PBK-AKT-mTOR), e.g. colon, cervix, endometrium, pancreas, large and small intestine, skin, prostate, head and neck, and lung tumors,
in the treatment of post natal clinical manifestations of RASopathies caused by germline mutations of PTPN11 (Noonan syndrome and Noonan syndrome with multiple lentigines, also called LEOPARD syndrome), such as hypertrophic cardiomyopathy, short stature and predisposition to certain malignancies, particularly JMML,
in cancer immunotherapy to avoid the tumor immune evasion mediated by SHP2’s activation of immune checkpoint pathways, such as the Programmed Cell Death 1 (PD-1) or signal-regulatory protein alpha (SIRPa)/CD47, thus modulating the immune response in cancer,
as a cytotoxin-associated gene A (CagA) competitor in H. /ⱼ7 /7-rnediated gastric carcinoma.

13. (canceled)

14. A peptide according to claim 1 comprising the sequence from N-terminus to C-terminus: X₋₂X₋₁ZX₁X₂X₃X₄X₅ (SEQ ID NO: 1) wherein
Z is phosphonodifluoromethyl phenylalanine (F₂Pmp), tyrosine or phosphotyrosine (pY);
X-₂ is a hydrophobic amino acid selected from the group consisting of: Leu, Ile, Val, Phe, Tyr, Trp and Met;
X₋₁ is any amino acid;
X₁ is a hydrophobic amino acid selected from the group consisting of: Ile, Leu, Val, Phe, Tyr, Trp and Met;
X₃ is a hydrophobic amino acid selected from the group consisting of: Leu, Ile, Val, Phe, Tyr, Trp and Met;
X₅ is a hydrophobic amino acid selected from the group consisting of: Trp, Ile, Val, Phe, Tyr, and Met; and
X₂ and X₄ are each independently Asp or Glu
wherein the amino acids may be each independently a natural or non-natural amino acid, such as Ca or N methylated, peptoids, beta amino acids or D amino acids.

15. A peptide according to claim 1 comprising the sequence from N-terminus to C-terminus:
X₋₂X₋₁ZX₁X₂X₃X₄X₅ (SEQ ID NO:1)
wherein
Z is phosphonodifluoromethyl phenylalanine (F₂Pmp), tyrosine or phosphotyrosine (pY);
X-₂ is a hydrophobic amino acid selected from the group consisting of: Leu, Ile, Val, Phe, Tyr, Trp and Met;
- X-X₁ is any amino acid;
X₁ is a hydrophobic amino acid selected from the group consisting of: Ile, Leu, Val, Phe, Tyr, Trp and Met;
X₃ is a hydrophobic amino acid selected from the group consisting of: Leu, Ile, Val, Phe, Tyr, Trp and Met;
X₅ is a hydrophobic amino acid selected from the group consisting of: Trp, Phe and Tyr; and
X₂ and X₄ are each independently Asp or Glu
wherein the amino acids may be each independently a natural or non-natural amino acid.

16. A peptide according to claim 1 comprising the sequence from N- terminus to C-terminus:
X₋₂X₋₁ZX₁X₂X₃X₄X₅ (SEQ ID NO: 1)
wherein
Z is phosphonodifluoromethyl phenylalanine (F₂Pmp), tyrosine or phosphotyrosine (pY) ;
X-₂ is selected from the group consisting of: Leu, Ile, Val, Phe, Tyr, Trp and Met;
X₋₁ is any amino acid;
X₁ is a hydrophobic amino acid selected from the group consisting of: Ile, Leu, Val, Phe, Tyr, Trp and Met;
X₃ is a hydrophobic amino acid selected from the group consisting of: Leu, Ile, Val, Phe, Tyr, Trp and Met;
X₅ is Trp; and
X₂ and X₄ are each independently Asp or Glu
wherein the amino acids may be each independently a natural or non-natural amino acid.

17. The peptide according to claim 1 wherein:
X-₂ is Leu and/or
X₋₁ is Asn and/or
X₁ is Ile and/or
X₂ is Asp and/or
X₃ is Leu and/or
X₄ is Asp and/or
X₅ is Trp.

18. The peptide according to claim 1 wherein Z is F₂Pmp.

19. The peptide according to claim 9 wherein the aminoacidic sequence which favors penetration inside cells is TAT, penetratin, oligo-Arg, transportan 10, said amino acidic sequence being linked to the N-terminus and/or the C-terminus of the peptide.

20. The peptide according to claim 7, wherein the N-terminus is acetylated or labeled with CF and/or the C- terminus is amidated.

21. The non-covalent complex according to claim 10 wherein the aminoacidic sequence which favors penetration inside cells is Pepl.
